(19) United States
(12) Patent Application Publication
(10) Pub. No.: US 2003/0108533 A1
(43) Pub. Date: Jun. 12, 2003

(54) Mediators of signal transduction

(76) Inventors: Wenlin Zeng, Union City, CA (US); Lawrence Stanton, Redwood City, CA (US); Haiyan Kong, Sunnyvale, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/307,019

(22) Filed: Nov. 26, 2002

Related U.S. Application Data
(62) Division of application No. 09/548,473, filed on Apr. 13, 2000.
(60) Provisional application No. 60/129,552, filed on Apr. 16, 1999.

(30) Foreign Application Priority Data
Apr. 11, 2000 (WO) PCT/US00/09488

(51) Int. Cl. A61K 48/00; C07H 21/04; C12P 21/02; C12N 9/12; C12N 15/74; C12N 15/85; C12N 5/08

(52) U.S. Cl. 424/93.21; 435/69.1; 435/194; 435/320.1; 435/366; 536/23.2; 435/455

(57) ABSTRACT

Polypeptides capable of regulating signal transduction, which preferably exhibit kinase activity, or antibodies against such polypeptides that inhibit the interaction of these polypeptides with other mediators of signal transduction, may be used in the identification, prevention or treatment of disease, preferably cardiac disease, in mammalian hosts. In addition, these polypeptides can facilitate the identification or isolation of additional mediators of signal transduction associated with disease, preferably cardiac disease, which in turn may also be used in the identification, prevention or treatment of disease, preferably cardiac disease, in mammals.

### RELATED APPLICATIONS

This application is a divisional application of U.S. patent application Ser. No. 9/548,473, filed Apr. 13, 2000, now U.S. Pat. No. ______, and claims the benefit of priority from U.S. Provisonal Patent Application No. 60/129,552, filed Apr. 16, 1999, both of which are hereby incorporated by reference in their entirety.

### FIELD OF THE INVENTION

The present invention relates to compounds and methods for the identification, prevention or treatment of disease, preferably cardiac disease, in a mammal through the administration of polypeptides capable of regulating signal transduction, which preferably exhibit kinase activity, or antibodies against such polypeptides that inhibit the interaction of these polypeptides with other mediators of signal transduction. In addition, the compounds and methods of the present invention can facilitate the identification or isolation of additional mediators of signal transduction associated with disease, preferably cardiac disease, which in turn may also be used in the identification, prevention or treatment of disease, preferably cardiac disease, in mammals.

### BACKGROUND OF THE INVENTION

Certain biological functions, such as growth and differentiation, are tightly regulated by signal transduction pathways within cells. Signal transduction pathways maintain the balanced steady state functioning of a cell. Disease states can arise when signal transduction in a cell breaks down, thereby removing the control that typically exists over cellular functions. Because signal transduction networks regulate a multitude of cellular functions depending upon the cell type, a wide variety of diseases can result from abnormalities in such networks. Devastating diseases such as cancer, autoimmune diseases, allergic reactions, inflammation, neurological disorders and hormone-related diseases can result from abnormal signal transduction. For example, tumors may develop when regulation of cell growth is disrupted.

Despite a long-felt need to understand and discover methods for regulating cells involved in various disease states, the complexity of signal transduction pathways has precluded the development of products and processes for regulating cellular function by manipulating signal transduction pathways in a cell. As such, there remains a need for products and processes that permit the implementation of predictable controls of signal transduction in cells, thus enabling the treatment of various diseases that are caused by abnormal cellular function.

Such diseases may include cardiac diseases, which may include congestive heart failure (CHF), dilated congestive cardiomyopathy, hypertrophic cardiomyopathy, restrictive cardiomyopathy, mitral valve disease, aortic valve disease, tricuspid valve disease, angina pectoris, myocardial infarction, cardiac arrhythmia, pulmonary hypertension, arterial hypertension, renovascular hypertension, arteriosclerosis, atherosclerosis, and cardiac tumors. By way of example, CHF is a major cardiac disease associated with extensive morbidity and mortality. Approximately five million individuals in the United States suffer from some form of CHF. Traditionally, treatment of CHF occurs by a series of agents including diuretics, vasodilators, angiotensin converting enzyme inhibitors, B-adrenergic antagonists, and positive inotropes like digoxin. These drugs, however, principally provide symptomatic relief and typically only extend the life of one suffering from the disease for periods ranging from 6-12 months.

The pathophysiology of CHF is rather complex. Generally, the central hallmark of the disease is the inability of the heart to pump sufficient oxygenated blood to meet the demands of peripheral tissues. Numerous etiologies contribute to the development of CHF, including primary diseases of, or insults to, the myocardium itself, cardiac defects, hypertension, inflammation, kidney disease and vascular disease. These conditions lead to the hypertrophy and remodeling of the cardiac ventricles which, if unchecked, ultimately reduce the mechanical performance of the heart. Forces associated with the inability of the heart to pump blood ultimately lead to the release of neurohormones like catecholamines, renin-angiotensin, aldosterone, endothelin and related factors into the circulation. Elevations in plasma levels of many of these circulating neurohormones have a deleterious impact on the outcome of patients with CHF. Local production of these neurohormonal factors in the heart is believed to contribute centrally to the disease. Thus, an important therapeutic strategy has been to block this neurohormonal axis contributing to the pathogenesis of this disease.

Factors known to contribute centrally to the pathophysiology of heart disease are biosynthesized in the heart itself. These factors are produced in cardiac myocytes, fibroblasts, smooth muscle and endothelial cells, and inflammatory cells associated with the myocardium. For example, the heart contains its own renin-angiotensin system. Blockade of the cardiac renin-angiotensin system may contribute significantly to the therapeutic efficacy of the therapeutic class of agents known as angiotensin converting enzyme (ACE) inhibitors.

The heart also produces other factors including endothelins, bradykinin, adrenomedullin, tumor necrosis factor, transforming growth factors, and natriuretic peptides. Unfortunately, therapeutic strategies are limited to the modulation of such substances, which are already known to contribute to the disease. Indeed, the functional contributions of only a minor fraction of all known secreted factors encoded by the human genome have apparently been defined.

The foregoing shows a need for methods and products involving the prevention or treatment of disease in mammals involving the mediation of signal transduction. The administration of polypeptides capable of regulating signal transduction, which preferably exhibit kinase activity, or antibodies against such polypeptides that inhibit the interaction of these polypeptides with other mediators of signal transduction, in addition to the identification or isolation of additional mediators of signal transduction associated with disease, preferably cardiac disease, which in turn may also be used in the identification, prevention or treatment of disease, preferably cardiac disease, in mammals, can facilitate such prevention or treatment.

### SUMMARY OF THE INVENTION

An objective of the present invention is therefore the prevention or treatment of disease, preferably cardiac disease, in mammals through the administration of polypeptides capable of regulating signal transduction, which preferably exhibit kinase activity, or antibodies against such polypeptides that inhibit the interaction of these polypeptides with other mediators of signal transduction, in addition to the identification or isolation of additional mediators of signal transduction associated with disease, preferably cardiac disease, which in turn may also be used in the identification, prevention or treatment of disease, preferably cardiac disease, in mammals.

In accomplishing these and other objectives, the present invention preferably provides a purified polypeptide comprising the amino acid sequence of SEQ ID NOS: 1, 4, 6, 7, 8 or 9 wherein the polypeptide is capable of regulating signal transduction. In a preferred embodiment, the polypeptide is capable of catalyzing the transfer of a phosphate group from a donor molecule to an acceptor molecule.

In another embodiment, the present invention preferably provides an isolated DNA molecule encoding a purified polypeptide comprising the amino acid sequence of SEQ ID NOS: 1, 4, 6, 7, 8 or 9 wherein the polypeptide is capable of regulating signal transduction. The present invention may also preferably be an isolated DNA molecule comprising the nucleotide sequence of SEQ ID NOS: 2, 3 or 5.

In yet another embodiment, the present invention preferably provides a vector comprising a DNA molecule encoding a purified polypeptide comprising the amino acid sequence of SEQ ID NOS: 1, 4, 6, 7, 8 or 9 wherein the polypeptide is capable of regulating signal transduction. In another aspect, the present invention provides a host cell transformed with such a vector. In one other embodiment, the present invention may preferably provide the above-described transformed host cell, where the host cell produces a polypeptide capable of regulating signal transduction. In a preferred embodiment, the above-described transformed host cell produces a polypeptide capable of catalyzing the transfer of a phosphate group from a donor molecule to an acceptor molecule.

In another preferred embodiment, the present invention provides an isolated antibody against a polypeptide comprising the amino acid sequence of SEQ ID NOS: 1, 4, 6, 7, 8 or 9 wherein the polypeptide is capable of regulating signal transduction. In a preferred embodiment, the antibody is a monoclonal antibody. In another preferred embodiment, the antibody is capable of inhibiting the regulation of signal transduction. In yet another preferred embodiment, the antibody is capable of inhibiting the transfer of a phosphate group from a donor molecule to an acceptor molecule.

The present invention may also preferably provide an isolated nucleic acid capable of hybridizing under high stringency conditions to a DNA molecule comprising the nucleotide sequence of SEQ ID NOS: 2, 3 or 5. In a preferred embodiment, this isolated nucleic acid is capable of inhibiting the regulation of signal transduction. In yet another preferred embodiment, this isolated nucleic acid is capable of inhibiting said transfer of said phosphate group from said donor molecule to said acceptor molecule.

In a preferred embodiment, the present invention provides a method of preventing or treating disease in a mammal comprising administering to said mammal an effective amount of material, selected from the group consisting of the polypeptide comprising the amino acid sequence of SEQ ID NOS: 1, 4, 6, 7, 8 or 9 wherein the polypeptide is capable of regulating signal transduction, and the antibody against this polypeptide, in a pharmaceutically acceptable sterile vehicle. In a preferred embodiment, the mammal may be a human. In another, the disease may be cardiac disease.

The present invention may also preferably provide a vaccine for preventing disease in a mammal comprising administering to said mammal an effective amount of material, selected from the group consisting of the polypeptide comprising the amino acid sequence of SEQ ID NOS: 1, 4, 6, 7, 8 or 9 wherein the polypeptide is capable of regulating signal transduction, and the antibody against this polypeptide, in a pharmaceutically acceptable sterile vehicle. In a preferred embodiment, the mammal may be a human. In another, the disease may be cardiac disease.

In a preferred embodiment, the present invention provides a method of preventing or treating disease in a mammal comprising administering to said mammal syngeneic cells transformed with a vector comprising a DNA molecule encoding a purified polypeptide comprising the amino acid sequence of SEQ ID NOS: 1, 4, 6, 7, 8 or 9 wherein the polypeptide is capable of regulating signal transduction, and wherein the transformed syngeneic cells produce a polypeptide capable of regulating signal transduction. In a preferred embodiment, the mammal may be a human. In another, the disease may be cardiac disease.

The present invention may also preferably provide a method of preventing or treating disease in a mammal comprising administering to said mammal syngeneic cells transformed with a vector comprising a DNA molecule encoding a purified polypeptide comprising the amino acid sequence of SEQ ID NOS: 1, 4, 6, 7, 8 or 9 wherein the polypeptide is capable of regulating signal transduction, and wherein the transformed syngeneic cells produce a polypeptide capable of catalyzing the transfer of a phosphate group from a donor molecule to an acceptor molecule. In a preferred embodiment, the mammal may be a human. In another, the disease may be cardiac disease.

In a preferred embodiment, the present invention may provide a kit for detecting the expression of a protein capable of regulating signal transduction, comprising a polypeptide, which comprises the amino acid sequence of SEQ ID NOS: 1, 4, 6, 7, 8 or 9 wherein the polypeptide is capable of regulating signal transduction. In a preferred embodiment, this kit further comprises a detectable label selected from the group consisting of calorimetric, enzymatic, chemiluminescent, fluorescent and radioactive labels.

In another preferred embodiment, the present invention may provide a kit for detecting the expression of a protein capable of acting as a donor molecule or an acceptor molecule of a phosphate group comprising a polypeptide, which comprises the amino acid sequence of SEQ ID NOS: 1, 4, 6, 7, 8 or 9 wherein the polypeptide is capable of regulating signal transduction. In a preferred embodiment, this kit further comprises a detectable label selected from the group consisting of calorimetric, enzymatic, chemiluminescent, fluorescent and radioactive labels.

The present invention may also preferably provide a method for detecting the expression of a protein capable of regulating signal transduction, comprising contacting a sample with a polypeptide, which comprises the amino acid sequence of SEQ ID NOS: 1, 4, 6, 7, 8 or 9 wherein the polypeptide is capable of regulating signal transduction, and detecting any effect of the sample on an indicator of signal transduction. In a preferred embodiment, the polypeptide is immobilized to a solid support. In another preferred embodiment, the phosphate group is detectably labeled.

In another preferred embodiment, the present invention may provide a method for detecting the expression of a protein capable of acting as a donor molecule or an acceptor molecule of a phosphate group, comprising contacting a sample with a polypeptide, which comprises the amino acid sequence of SEQ ID NOS: 1, 4, 6, 7, 8 or 9 wherein the polypeptide is capable of regulating signal transduction, and detecting any effect of the sample on an indicator of signal transduction, and detecting any transfer of the phosphate group. In a preferred embodiment, the polypeptide is immobilized to a solid support. In another preferred embodiment, the phosphate group is detectably labeled.

Other objectives, features, and advantages of the present invention will become apparent from the following detailed description. The detailed description and the specific examples, while indicating preferred embodiments of the invention, are provided by way of illustration only. Accordingly, the present invention also includes those various changes and modifications within the spirit and scope of the invention that may become apparent to those skilled in the art from this detailed description.

### BRIEF DESCRIPTION OF THE FIGURES

FIG. 1 illustrates eight overlapping human cDNA clones of 19G5.

FIG. 2 depicts homology alignment of amino acid sequences of human, rat and mouse clones corresponding to 19G5.

FIG. 3 depicts the likely gene structure of human 19G5 (H19G5), which reveals at least 10 exons and 9 introns.

FIG. 4 is a schematic diagram of four cDNA clones corresponding to splicing variants of H19G5. The longest clone (C11) contains two kinase domains, the N-terminal and the C-terminal kinase domains (N-KD and C-KD).

FIG. 5 is a schematic diagram comparing the domain structure of 19G5 and Trio proteins.

FIG. 6 is the sequence comparison of the kinase domains of H19G5 (N-terminal and C-terminal kinase domains), Trio, and smooth muscle myosin light chain (SM MLCK).

FIG. 7 is the sequence comparison of the guanine nucleotide exchange factor (GEF) domains of H19G5 and Trio.

FIG. 8 shows the subcellular localization of 19G5-GFP fusion proteins in mouse myoblast cell line C2C12. Three 19G5-GFP fusion protein expression constructs were made using three different 19G5 cDNA clones, the longest human 19G5 clone C11 [h19G5(C11)-GFP], a 2.7 kb clone of human 19G5 containing the C-terminal kinase domain [h19G59F1)-GFP], and the rat 19G5 small transcript [r19G5(S)-GFP]. The control GFP vector and the 19G5-GFP fusion expression constructs were transfected into C2C12 cells. The 19G5-GFP fusion proteins' localization was detected using confocal microscopy.

FIG. 9 shows that H19G5 protein expressed by the clone C11 binds to the small G protein Cdc42. The lysate of 293 EBNA cells transfected with H19G5-C11 expression construct was incubated with GST-Cdc42 immobilized on glutathione-agarose. After washing, the complex was resuspended in SDS sample buffer, boiled and run on a SDS-PAGE, and Western blotted with an anti-H19G5 monoclonal antibody.

FIG. 10 shows phase contrast micrographs of C2C12 myoblasts undergoing differentiation into myotubes. Undifferentiated C2C12 cells are shown in Day 0. Differentiation of C2C12, induced by placing in 2% horse serum, is apparent on Day 3 and Day 5 as shown.

FIG. 11 shows induction of myogenin protein expression when C2C12 myoblast cells are induced to differentiate into myotubes. Myogenin is a marker of myotubes.

FIG. 12 shows induction of 19G5 RNA expression during the differentiation of myoblasts into myotubes.

FIG. 13 shows inhibition of the induction of 19G5 expression by TFG-β during C2C12 differentiation.

### DETAILED DESCRIPTION OF THE PREFERRED EMBODIMENTS

Those skilled in the art will recognize that the products and methods embodied in the present invention may be applied to a variety of systems, constructed with various materials using various methods. Accordingly, the present invention is not limited to any particular environment, and the following description of specific embodiments of the present invention are for illustrative purposes only.

The present invention preferably provides methods for the prevention or treatment of disease, preferably cardiac disease, in mammals through the administration of polypeptides capable of regulating signal transduction, which preferably exhibit kinase activity, or antibodies against such polypeptides that inhibit the interaction of these polypeptides with other mediators of signal transduction, in addition to the identification or isolation of additional mediators of signal transduction associated with disease, preferably cardiac disease, which in turn may also be used in the identification, prevention or treatment of disease, preferably cardiac disease, in mammals. The cardiac diseases according to the present invention may include congestive heart failure (CHF), dilated congestive cardiomyopathy, hypertrophic cardiomyopathy, restrictive cardiomyopathy, mitral valve disease, aortic valve disease, tricuspid valve disease, angina pectoris, myocardial infarction, cardiac arrhythmia, pulmonary hypertension, arterial hypertension, renovascular hypertension, arteriosclerosis, atherosclerosis, and cardiac tumors.

An embodiment of the invention is a purified polypeptide comprising an amino acid sequence having at least 95% sequence identity with the amino acid sequence of SEQ ID NOS: 1, 4, 6, 7, 8 or 9. As used herein, polypeptide refers to a linear series of amino acid residues connected to one another by peptide bonds between the alpha-amino groups and carboxy groups of adjacent amino acid residues. Additional covalent bonds between portions of the peptide may also be present to restrain the conformation of the molecule, such as amide and disulfide bonds. When used herein, polypeptide also refers to a linear series of amino acid residues connected one to the other as in a peptide. The term synthetic peptide means a chemically derived chain of amino acid residues linked together by peptide bonds that is free of naturally occurring proteins and fragments thereof.

The one and three-letter symbols used to represent the amino acid residues in the polypeptides of the present invention are those symbols commonly used in the art. The amino acid residues are preferred to be in the L isomeric form. However, residues in the D isomeric form may be substituted for any L-amino acid, as long as the desired functional property of signal transduction mediation is retained by the peptide. The one and three-letter symbols used herein refer to the following amino acids: Ser (S) is serine; Ile (I) is isoleucine; Gln (Q) is glutamine; Phe (F) is phenylalanine; His (H) is histidine; Trp (W) is tryptophan; Lys (K) is lysine; Asn (N) is asparagine; Leu (L) is leucine; Gly (G) is glycine; Thr (T) is threonine; Asp (D) is aspartic acid; Arg (R)is arginine; and Ala (A) is alanine.

Polypeptides of the present invention include variants, fragments and chemical derivatives of the polypeptides comprising the amino acid sequence of SEQ ID NOS: 1, 4, 6, 7, 8 or 9 as long as they are capable of mediating signal transduction. Polypeptides thus may include soluble peptides, Ig-tailed fusion peptides (including immunoadhesions), members of random peptide libraries (see, e.g., Lam, K. S. et al., Nature 354:82-84 (1991); Houghten, R. et al., Nature 354:84-86 (1991)), combinatorial chemistry-derived molecular libraries made of D-and/or L-configuration amino acids, and phosphopeptides (including members of random or partially degenerate, directed phosphopeptide libraries, see, e.g., Songyang, Z. et al., Cell 72:767-778(1993)).

Polypeptides of the present invention may also include polypeptides that can be isolated from nature or can be produced by recombinant and/or synthetic means. Such native sequence polypeptides specifically refers to naturally-occurring truncated or secreted forms (e.g., an extracellular domain sequence), as well as naturally occurring variant forms (e.g., alternatively spliced forms), and naturally occurring allelic variants of the named polypeptides.

The term variant refers to any polypeptide having an amino acid sequence, in comparison to the amino acid sequences of the polypeptides of the present invention, in which one or more amino acids have been substituted with other amino acids; where the substituted amino acids allow or require the polypeptide to assume the equilibrium conformation of the domain of the parent protein. Often, cysteine, lysine and glutamic acid will be used for their side chains which can form covalent linkages to restrict the conformation of a peptide. The term variant refers to any polypeptide in which one or more amino acids are added and/or substituted and/or deleted and/or inserted at the N- or C-terminus or anywhere within the corresponding native sequence, and which retains signal transduction mediation activity of the corresponding native polypeptide. The variants herein preferably comprise a sequence that has at least about 80% sequence identity, more preferably at least about 85% sequence identity, even more preferably at least about 90% sequence identity, most preferably at least about 95% sequence identity, with the amino acid sequence of SEQ ID NOS: 1, 4, 6, 7, 8 or 9.

In such amino acid sequences, one or more amino acids in the fundamental sequence may preferably be substituted with another amino acid(s), the charge and polarity of which is similar to that of the native amino acid, i.e., a conservative amino acid substitution, resulting in a silent change. Substitutes for an amino acid within the fundamental polypeptide sequence can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cyteine, cystine, tyrosine, asparagine, and glutamine; (4) neutral nonpolar (hydrophobic) amino acids such as alanine, leucine, isoleucine, valine, proline, phenylalanine, tryptophan, and methionine.

The term variant shall also include any polypeptide having one or more amino acids deleted from or added to an amino acid sequence of a mediator of signal transduction, but which still retains signal transduction mediation activity. The term fragment shall refer to any shorter version of the polypeptides herein, wherein the fragment is capable of mediating signal transduction.

Sequence identity is defined as the percentage of amino acid residues in a candidate sequence that are identical with the amino acid residues in a native polypeptide sequence, after aligning the sequences and introducing gaps, if necessary, to achieve the maximum percent sequence identity, and not considering any conservative substitutions as part of the sequence identity. The % sequence identity values are preferably generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1. Other algorithms, such as GAP, BESTFIT, FASTA, and TFASTA in the Wisconsin Genetics Software Package (Genetics Computer Groups, 575 Science Dr., Madison, Wis.), are also suitable. The selection of the non-default parameters to achieve maximum sequence identity is well within the skill of a person skilled in the art.

Antibodies of the present invention may include any polyclonal, monoclonal, humanized, anti-idiotypic, chimeric or single chain antibodies, and FAb, F(ab′)₂ and FAb fragments, and epitope-binding fragments thereof.

Without further elaboration, one skilled in the art with the preceding description can utilize the present invention to its fullest extent. The following examples are illustrative only, and not intended to limit the remainder of the disclosure in any way.

### EXAMPLE 1

### Isolation and Characterization of 19G5 Clones from Rat, Mouse and Human

Isolation of Partial and Full-Length 19G5 Clones from Rat, Mouse and Human

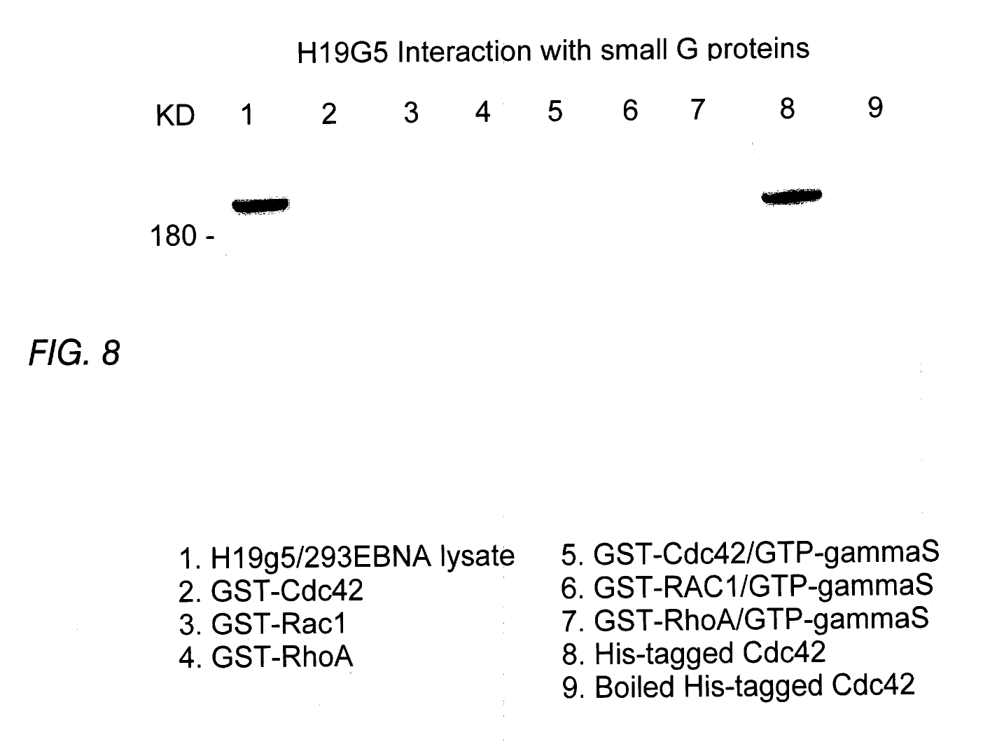

Microarray technology was used to identify genes that are differentially expressed in normal and diseased rat heart. A sequence designated as 19G5 was down regulated in 12-week myocardial infarct (MI) rat hearts. A rat cDNA clone corresponding to 19G5 (R19G5) was isolated and nucleotide sequence determined. The deduced amino acid sequence of the clone revealed homology to the catalytic domain of kinases, thus suggesting that the protein product of R19G5 might be involved in signal transduction. A hybridization of multiple tissue Northern blot with the R19G5 probe showed that the gene is highly expressed in heart as a 2 kb and 4.4 kb transcripts (FIG. 8).

A full length cDNA for the 2 kb R19G5 transcript was cloned using 5′ RACE technique. The R19G5 has an open reading frame of 1644 base pairs which encodes a protein of 548 amino acids (SEQ ID NO: 8)

A mouse 19G5 (M19G5) EST clone was identified by searching the EST database. Northern blot hybridization using the M19G5 EST clone as probe detected a major transcript of 2 kb in heart. There is also a low level of expression in lung. The smeary hybridization was also detected as in the rat and human but was less prominent. M19G5 was also expressed in 17-day old mouse embryos suggesting that it may play a role in embryo development. Sequence analysis of the M19G5 clone showed that it is 1900 base pairs long and has an open reading frame of 1644 base pairs coding for a polypeptide of 548 amino acids (SEQ ID NO: 9), suggesting that it likely represents full length 2 kb transcript.

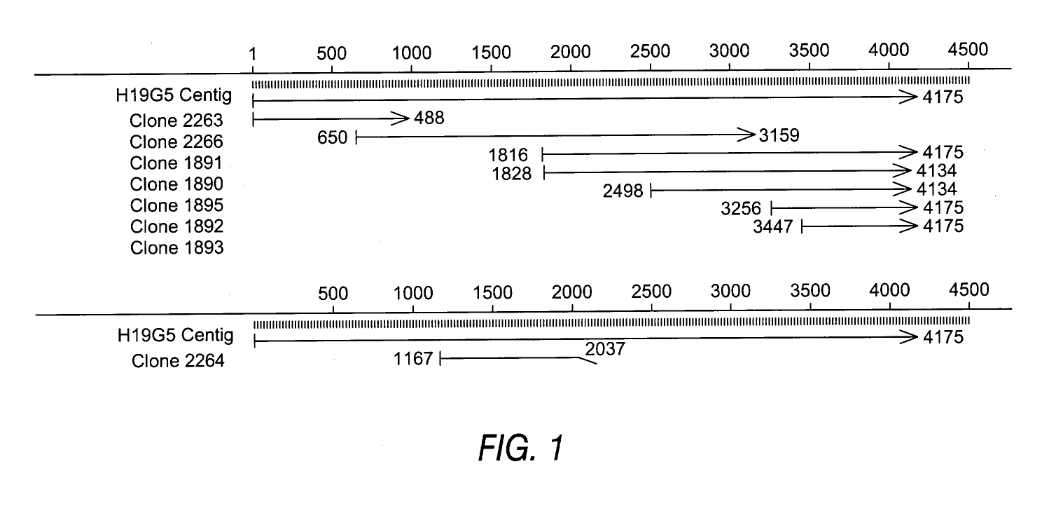

A number of human cDNA clones corresponding to 19G5 (H19G5) were isolated from human CDNA library using R19G5 as a probe. FIG. 1 shows eight overlapping cDNA clones of H19G5. Extensive overlap among these clones helped build a consensus nucleotide sequence (SEQ ID NO: 2) designated as H19G5 contig. The sequence corresponds to a major transcript (˜5 kb) expressed in human heart. The sequence of the contig revealed that it is complete at the 3′-end since it contains a polyadenylation signal (AATAAA) as well as polyA residues at the 3′-end. It has a potential open reading frame, coding for 1351 amino acid residues, that extends beyond the 5′-end of the contig indicating that it is incomplete at the 5′-end. The deduced amino acid sequence (SEQ ID NO: 1) revealed a protein kinase domain at the C-terminal end (amino acid residues 1056 to 1309) and also a partial protein kinase domain towards the N-terminal end (amino acid residues 1 to 105) of this truncated clone. The sequence information of this contig (SEQ ID NO: 2) was used to devise antisense primers corresponding to the 5′-end, which were used in 5′ RACE (rapid amplification of cDNA ends) to isolate cDNA clones with longer inserts. A cDNA clone containing full-length coding sequence was isolated. The nucleotide sequence of the clone (SEQ ID NO: 3) revealed an open reading frame that could potentially code for 1667 amino acids long full-length polypeptide (SEQ ID NO: 4).

Using the 5′ RACE technique, the full coding region for one of the larger H19G5 (3 kb or 5 kb) transcripts was cloned. It has an open reading frame of 2613 base pairs encoding a protein of 811 amino acids.

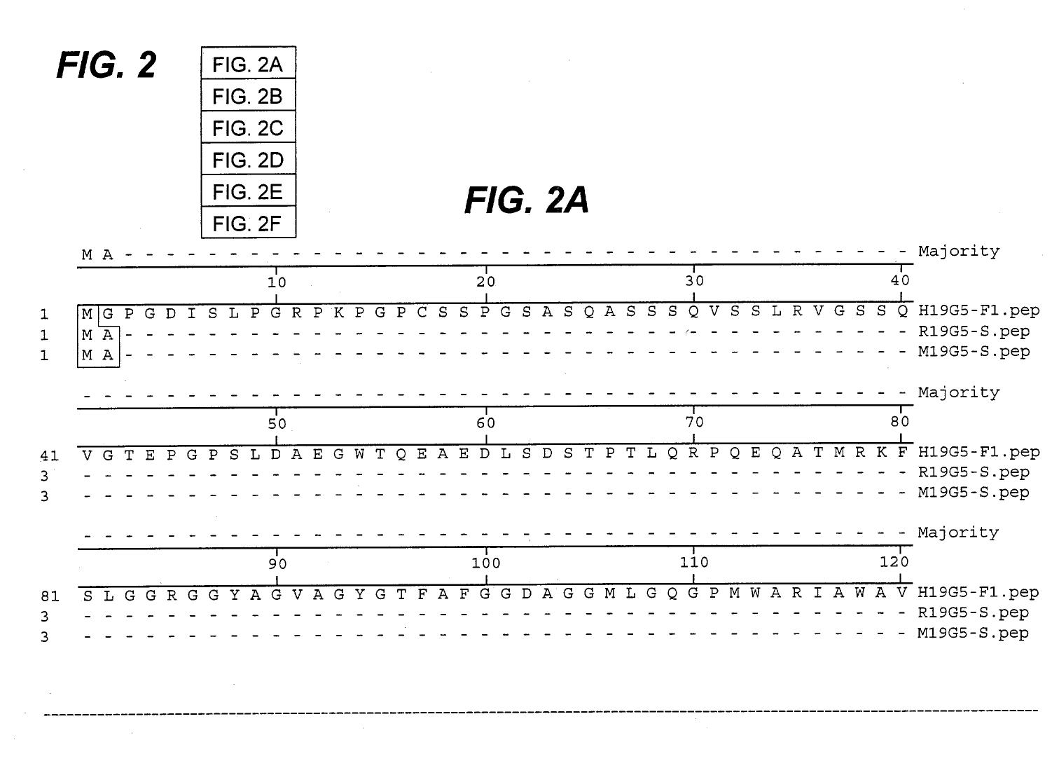

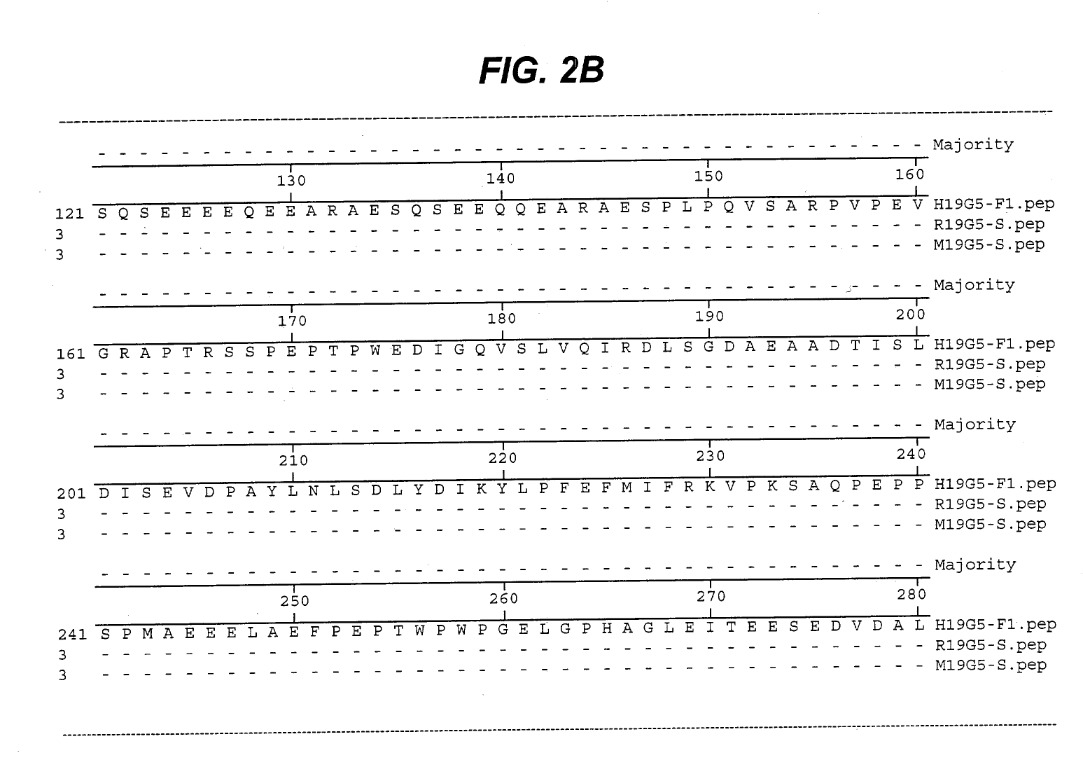

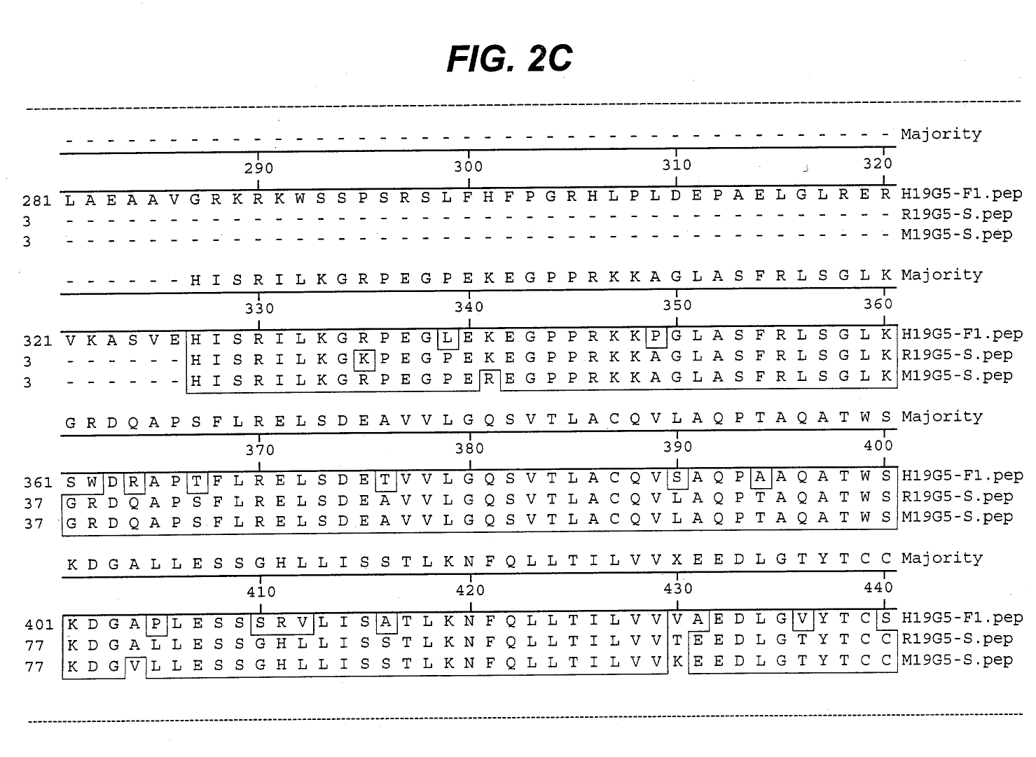

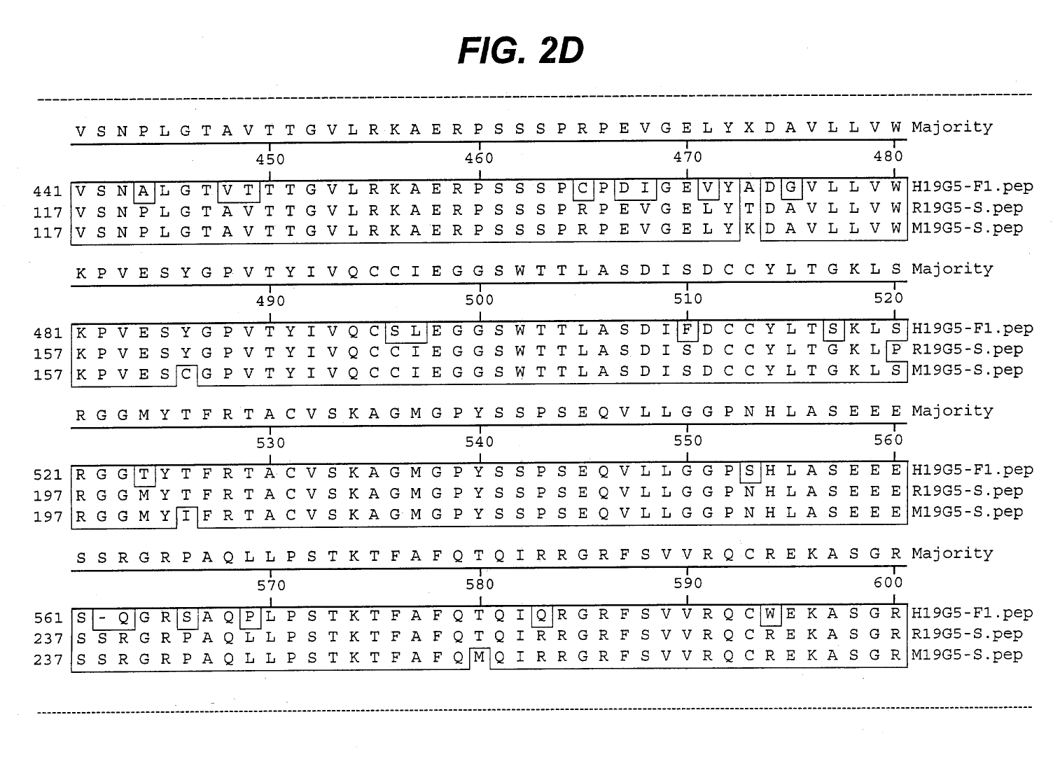

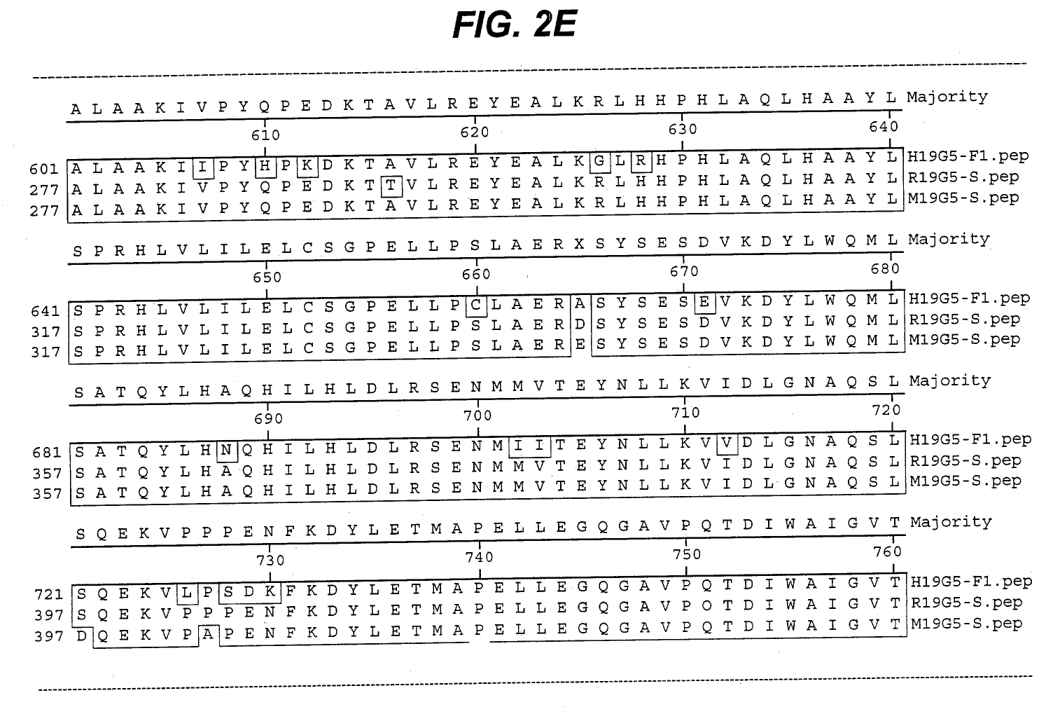

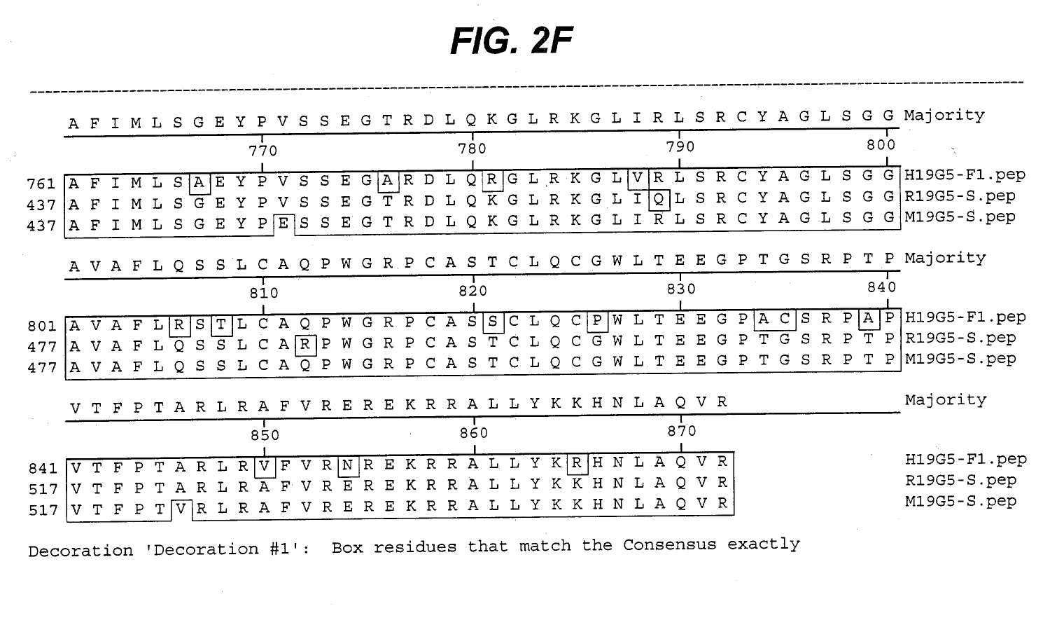

FIG. 2 shows the amino acid sequence alignment of the three full-length 19G5 proteins. Both the R19G5 (SEQ ID NO: 8) and M19G5 (SEQ ID NO: 9) proteins overlap with the C-terminal two-third of a splicing variant of the H19G5 protein (SEQ ID NO: 7). The identity between the R19G5 and M19G5 proteins is 97%. The three proteins are 85% identical in their sequences.

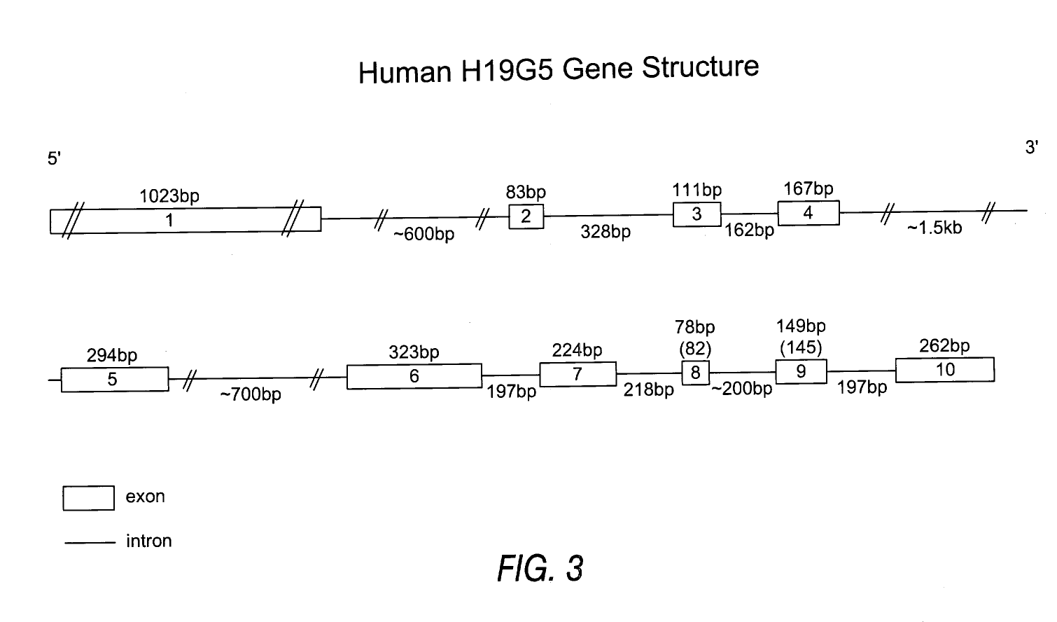

In order to understand the genomic organization of H19G5 gene, phage genomic library was screened and eleven H19G5 genomic clones were isolated. Restriction enzyme mapping of these clones detected no polymorphism, suggesting that H19G5 may be a single copy gene. The entire genomic DNA region encompassing H19G5 was sequenced and found to contain at least 10 exons and 9 introns as shown in FIG. 3.

One of the H19G5 cDNA clones contained deletion of two amino acid residues (alanine and proline) in the kinase domain. Sequence analysis of the genomic H19G5 DNA revealed an intron located immediately upstream of the alanine residue. There are two adjacent splicing acceptor sites that are four nucleotides apart at the 3′ end of the intron. A splicing event using the first acceptor site generates a protein that includes the two residues, alanine and proline. Utilization of the second splicing acceptor site creates a H19G5 protein with the two amino acids deleted in its kinase domain. These two residues are highly conserved among all kinases. This deletion form of H19G5 protein may thus exhibit reduced or no kinase activity at all, and may serve as a dominant negative inhibitor of the kinase activity of H19G5. This provides a possible mechanism to regulate the activity of H19G5 in vivo.

Isolation of cDNA Clones of Splicing Variants of H19G5

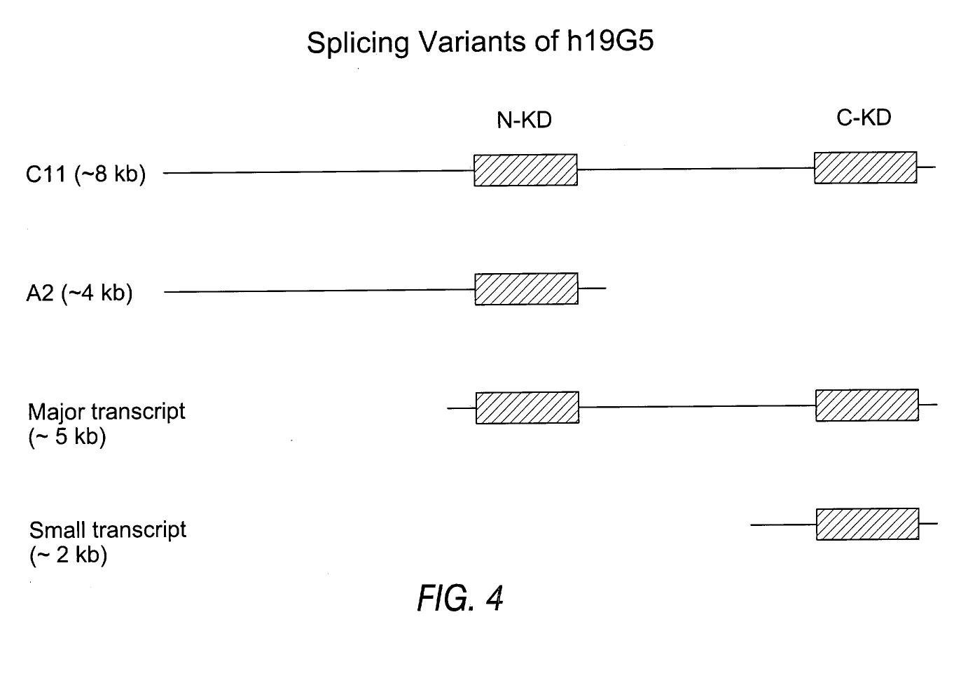

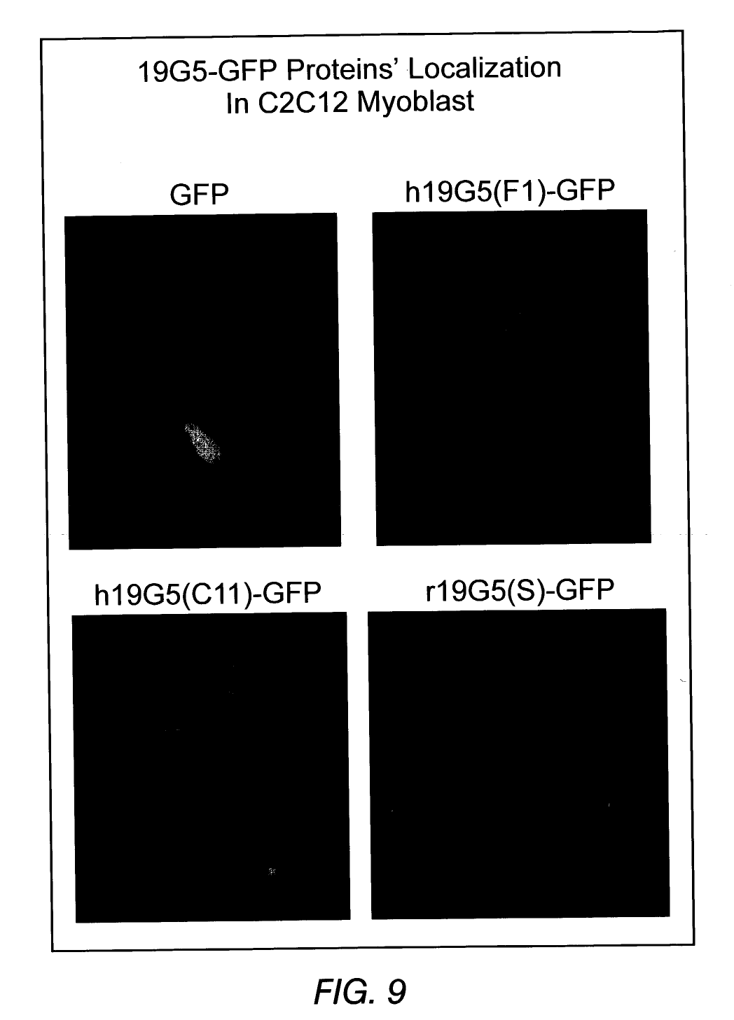

Multiple transcripts were detected in Northern analysis of human and rat cells, particularly in heart tissues (As shown in FIGS. 8 and 9). However, the restriction analysis of the genomic clones and Southern analysis of genomic DNA suggested single-copy nature of H19G5 gene. These observations indicated the possibility of alternative splicing as a source of multiple transcripts. Four cDNA clones representing various splicing variants were isolated and are schematically shown in FIG. 4. Complete nucleotide sequence (SEQ ID NO: 5) of the longest clone C11 was determined and the amino acid sequence of a large open reading frame contained therein was deduced (SEQ ID NO: 6). The large ORF has a potential to encode a protein of 2596 amino acid residues. The sequence analysis revealed the presence of a number of structurally and functionally important domains in H19G5. The presence of some of the domains strengthens the possibility of the involvement of H19G5 in signal transduction. For example, H19G5 has two kinase domains, one at the N-terminal (1094 to 1351 amino acid residues, N-KD) and the other at the C-terminal (2301 to 2553 amino acid residues, C-KD) end. H19G5 also has a Guanine nucleotide Exchange Factor (GEF) domain (325 to 504 amino acid residues), and a Pleckstrin Homology (PH) domain (532 to 634 amino acid residues). Additionally, H19G5 contains five immunoglobulin (Ig) like domains distributed throughout the sequence.

Structural and Functional Features of Full-Length H19G5 Protein Sequence

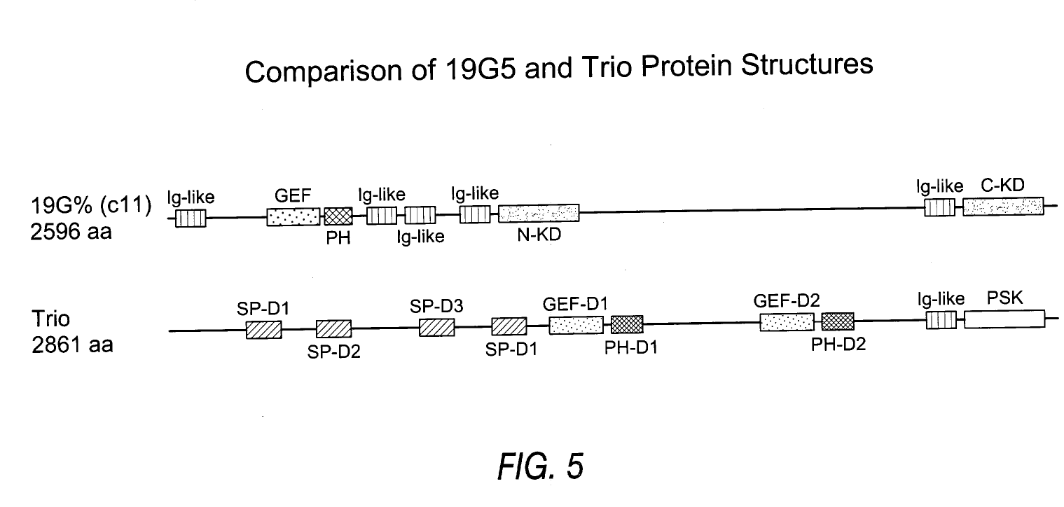

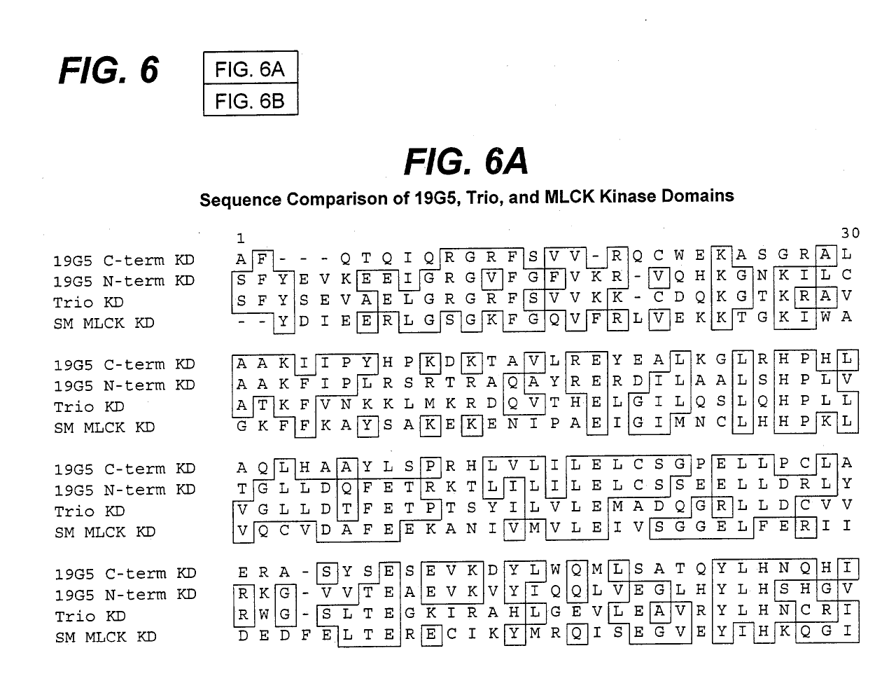

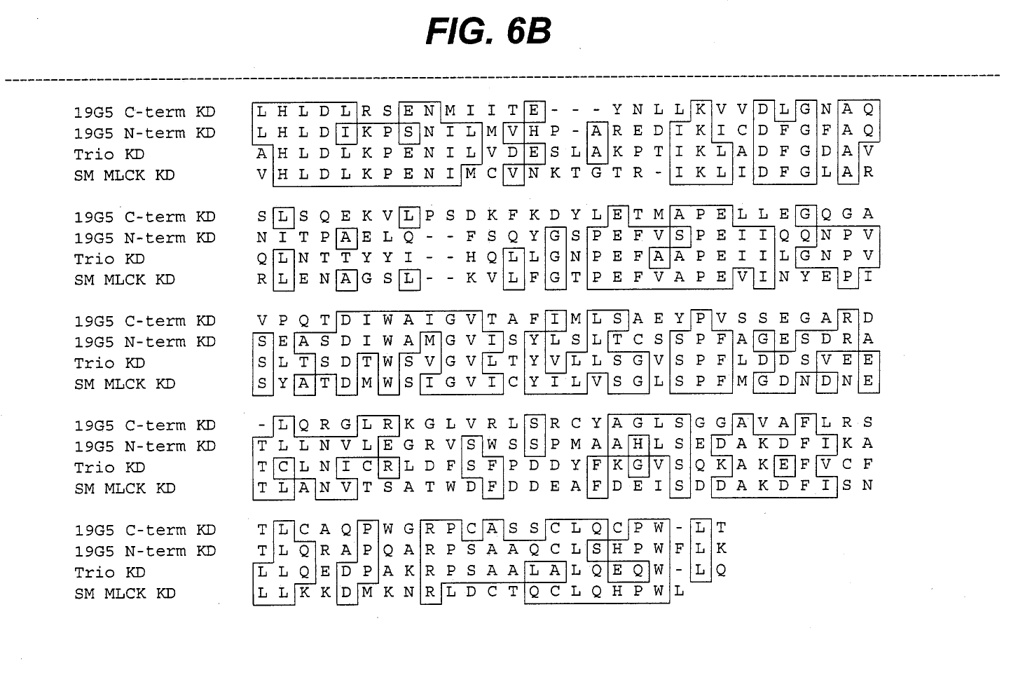

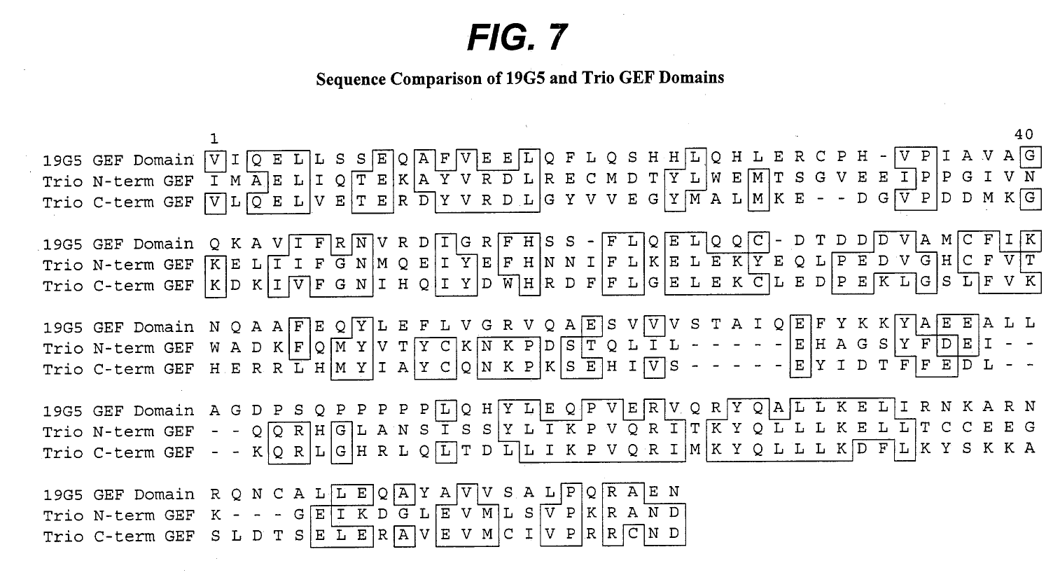

The full-length H19G5 amino acid sequence (SEQ ID NO: 6) shows sequence similarity to some functionally important domains of a protein called Trio. The comparison of the domain structure of H19G5 and Trio is schematically depicted in FIG. 5. Both kinase domains of H19G5 (N-KD and C-KD) are homologous to a single kinase domain of Trio as well as to a kinase domain of smooth muscle myosin light chain kinase (SM MLCK) as shown in FIG. 6. The identity between H19G5 N-KD and Trio KD is about 40% and between H19G5 N-KD and SM MLCK is about 38%. The sequence identity between the two kinase domains of H19G5 is about 30%. A single GEF domain of H19G5 is homologous to both GEF domains (GEF-D 1 and GEF-D2) of Trio as shown in FIG. 7.

Proteins containing GEF domains are involved in signal transduction (for a review, see Cherfils and Chardin, Trends Biochem. Sci. 24: 306-311[1999]). GEF domains promote exchange of GTP for GDP on GTP-binding proteins (G proteins) and thereby positively regulate their activities. As these proteins harbor intrinsic GTPase activity, they are also referred to as GTPases. These small G proteins, as opposed to trimeric G proteins, belong to a superfamily of Ras-like proteins. These proteins are bound to the inner face of plasma membrane, and usually exist in GDP-bound “inactive” state. When a ligand interacts with a membrane bound receptor, alteration of conformation allows the receptor to interact with a G protein. This interaction results in conformational change in the G protein that weakens the affinity for GDP and leads to replacement of GDP with GTP. This nucleotide exchange is greatly accelerated or promoted by proteins containing GEF domains. Once bound to GTP, the G proteins assume an “active” state in which they interact with the downstream effectors and facilitate transduction of signal from membrane to the nucleus. However, the activity of the G proteins is tightly controlled as their intrinsic GTPase activity rapidly hydrolyzes bound GTP into GDP and restores “inactive” status. Thus, G proteins function as molecular switches in signal transduction. A number of membrane receptors operate through G proteins. The downstream effectors of the activated G proteins include various protein kinases constituting a cascade of protein phosphorylation that brings about a desired change in gene expression.

As discussed in the preceding section, GEF domains play a critical role in signal transduction by controlling the activation of G proteins. Trio is a complex protein possessing two GEF domains, each with adjacent pleckstrin homology (PH) domains and Src Homology-3 (SH3) domains, a protein serine/threonine kinase domain with an adjacent immunoglobulin-like domain and multiple spectrin-like domains (Medley et al., J. Cell Sci. 112: 1825-1834[1999]). Trio CDNA clone was isolated by virtue of its ability to interact with protein tyrosine phosphatase (PTP) domain of a protein called LAR (Debant et al., Proc. Natl. Acad. Sci. USA 93: 5466-5471[1996]). LAR is a broadly expressed transmembrane protein tyrosine phosphatase comprised of a cell adhesion-like extracellular region and two intracellular PTPase domains, and is proposed to regulate cell-matrix interactions (Mourey and Dixon, Curr. Opin. Gen. Dev. 4: 31-39[1994]). Trio represents a unique member of the Rho-GEFs family possessing two functional GEF domains of distinct specificities. For example, GEF1 is specifically active on Rac1 GTPase, while GEF2 targets RhoA GTPase (Debant et al, supra). This unique feature allows Trio to link Rho and Rac specific signalling pathways in vivo.

The Rho family of Ras-like GTPases includes Rac (1, 2 and 3), RhoG, Cdc42Hs, TC10, TTF/RhoH, Rho (A, B and C), RhoD, RhoE, and RhoL. These proteins and other Ras-like proteins constitute Ras superfamily of structurally and functionally related GTPase proteins. These proteins are involved in diverse physiological functions such as control of cell shape (reviewed in Tapon and Hall, Curr. Opin. Cell Biol. 9: 86-92[1997]), cell motility (Aepfelbacher et al., Proc. Natl. Acad. Sci. USA 91: 4263-4267[1994]; and Curr. Biol. 6: 70-75[1996]), cell polarity (Adams et al, J. Cell Biol. 111: 131-142[1990]), smooth muscle contraction (Hirata et al., J. Biol. Chem. 267: 8719-8722[1992]), cell adhesion (Nobes and Hall, Cell 81: 53-62[1995]; Braga et al., J. Cell Biol. 137: 1421-1431 [1997]), cell division (Dutartre et al., J. Cell Sci. 109:367-377[1996]), vesicular transport between organelles such as receptor-mediated endocytosis (Lamaze et al., Nature 382: 177-179[1996]), apoptosis (Esteve et al., Oncogene 11: 2657-2665[1995]; Jimenez et al., Oncogene 10: 811-816[1995]; Gulbins et al., J. Biol Chem. 271: 26389-26394[1996]; Moorman et al., J. Immunol. 156: 4146-4153[1996]; Brenner et al., J. Biol. Chem. 272: 22173-22181[1997]) and normal and pathological cell proliferation (Olson et al., Science 269: 1270-1272[1995]; Hirai et al., J. Biol. Chem. 272: 13-16[1997]; Khosravi-Far et al., Mol. Cell. Biol. 16: 3923-3933[1996]; Qiu et al., Mol. Cell. Biol. 17: 3449-3458[1997]; Roux et al., Curr. Biol. 7: 629-637[1997]).

The presence of a GEF domain and protein kinase domains along with its homology to Trio suggests that H19G5 may possess guanine nucleotide exchange factor activity and protein kinase activity, both of which are shared by a number of proteins involved in signal transduction.

Expression Pattern of 19G5 in Rat and Human Tissues

Northern blot analysis revealed that R19G5 gene is highly expressed in heart as a 2 kb and a 4.4 kb transcripts (FIG. 8). Hybridization was performed using multiple tissue Northern blot (Clontech, Palo Alto, Calif.) and ExpressHyb solution following the manufacturer's protocol. The R19G5 probe also hybridized to mRNAs from skeletal muscle and detected multiple weak bands. High background observed in a lane corresponding to skeletal muscles is not due to RNA degradation as probing of the same blot with β-actin probe detected the right sized transcripts with a clean background (FIG. 8). The significance of the high background in skeletal muscle is not clear. Expression of R19G5 was not detected in brain, kidney, spleen, lung, liver, and testis.

Hybridization of human multiple tissue Northern blot (Clontech, Palo Alto, Calif.) with H19G5 probe detected a strong transcript of about 5 kb and three minor transcripts of about 3 kb, 2.4 kb, and 1.8 kb in heart tissue (FIG. 9). There were multiple transcripts and high background in skeletal muscle as seen with rat tissue. Only the 3 kb transcript was detected in human fetal heart. There is a low level expression of the 2.4 kb transcript in brain. H19G5 expression was not detected in spleen, lung, liver, kidney, pancreas, thymus, prostates, testis, ovary, small intestine, colon, peripheral blood leukocyte, stomach, thyroid, spinal cord, lymph node, trachea, adrenal gland, bone marrow, uterus muscle, or bladder muscle by Northern blot.

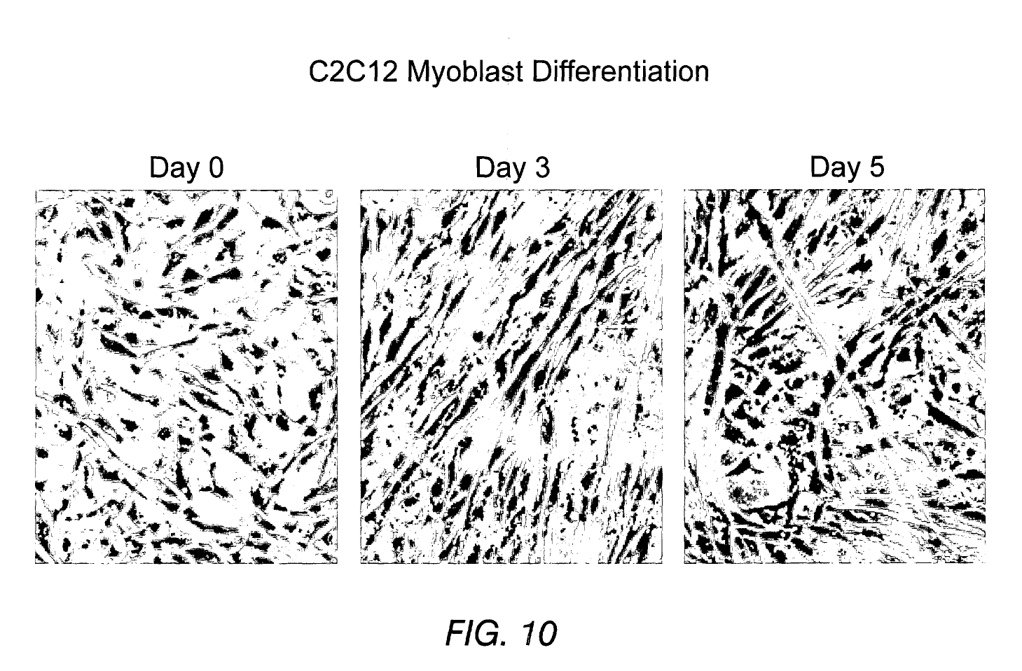

In order to determine whether the multiple transcripts of 19G5 in human heart are derived from alternative splicing or multiple gene copies, Southern blot analysis of genomic DNA was performed with H19G5 probe (FIG. 10). The results suggest that H19G5 is a single copy gene. This is consistent with the lack of detection of polymorphism in the restriction analysis of the genomic clones of H19G5.

Functional Characterization of H19G5 Protein

A 20 amino acid peptide from the C-terminus of H19G5 protein (SEQ ID NO: 1) was used to raise polyclonal antibodies in rabbits. Affinity purified rabbit anti-H19G5 antiserum has been obtained. Two different GST-H19G5 fusion proteins were used as antigens to generate mouse monoclonal anti-H19G5 antibodies. One of the antigens used was a GST-H19G5 fusion protein containing amino acid residues 610 to 811 of SEQ ID NO: 1. Many clones of anti-19G5 monoclonal antibodies were obtained. Three of them were shown to recognize recombinant H19G5 proteins expressed in mammalian cells by Western analysis and immunocytochemistry. The antibodies may be used to determine the size and localization of 19G5 protein by Western blot and immunohistochemistry.

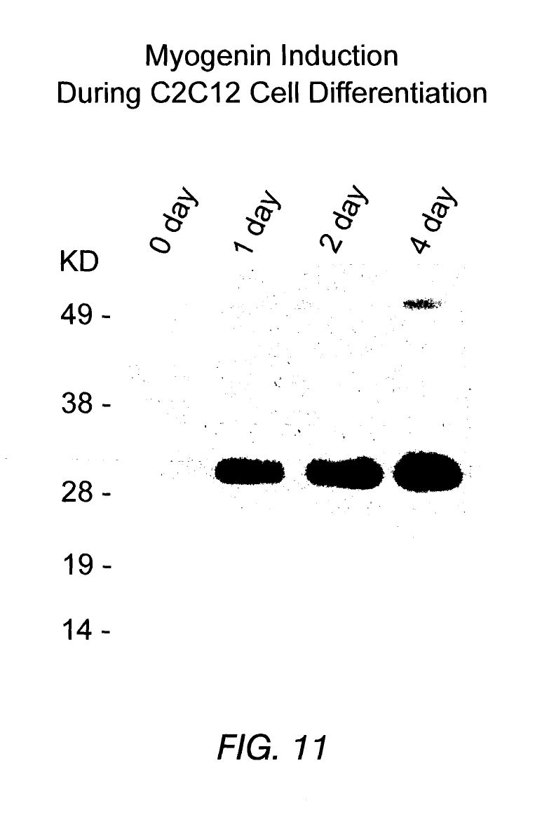

Subcellular localization of H19G5 protein was determined using confocal microscopy on cells transfected with vectors expressing 19G5 proteins fused to Green Fluorescent Protein (GFP). Three 19G5-GFP fusion protein expression constructs were made using three different 19G5 cDNA clones, the longest human 19G5 clone C11 [H19G5(C11)-GFP], a 2.7 kb clone of human 19G5 containing the C-terminal kinase domain [H19G59F1)-GFP], and the rat 19G5 small transcript [R19G5(S)-GFP]. The control GFP vector and the 19G5-GFP fusion expression constructs were transfected into C2C12 cells (ATCC Catalog No. CRL-1772). Cells were grown on chamber slides and transfected using SuperFectamine reagent from Quiagen for 3 hrs. Cells were fixed 24 hrs post-transfection with 4% paraformaldehyde for 15 min at room temperature and examined using a confocal microscope. The two longer forms of human 19G5-GFP proteins were detected in the nuclei whereas the short rat 19G5-GFP fusion protein was detected in the cytoplasm (FIG. 11). This suggests that different forms of 19G5 proteins are localized in different regions in the cells and may have different functions. Since the gene is normally expressed in heart, the localization of 19G5 protein in cardiac myocytes may be examined. Antisense constructs to study the effects of inhibiting the activity of 19G5 protein on cardiac myocytes may also be made and used.

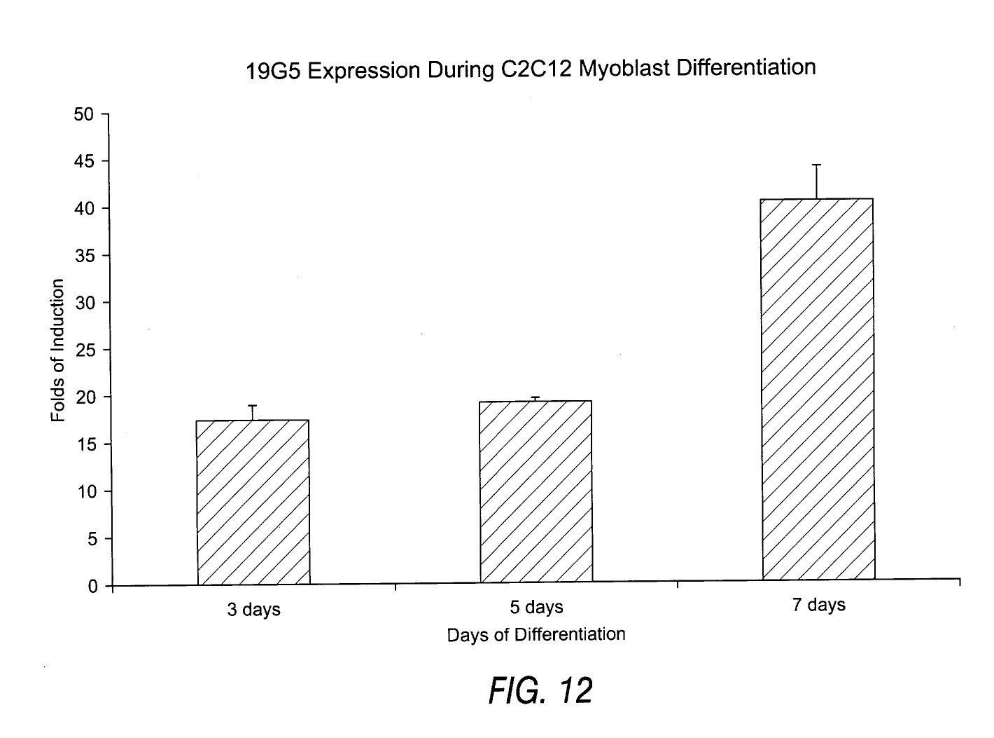

A possibility that H19G5 might interact with various G proteins, as suggested by the presence of GEF domain and homology with Trio, was examined. HEK 293 cells constitutively expressing Epstein-Barr Virus Nuclear Antigen (EBNA) (Invitrogen, San Diego, Calif.) were transfected with H19G5-C11 expression construct. The eukaryotic vector used for 19G5 expression, pEAK8 (EdgeBiosystems), contains Epstein-Barr virus (EBV) origin of DNA replication, which allows replication of the expression construct in transfected cells thereby amplifying the level of protein expression. After 24 hrs, one 10 cm dish of transfected 293 EBNA cells were lysed with 1 ml of lysis buffer (1X PBS, 0.1% Triton, and proteinase inhibitors (0.2 mM AEBSF, 0.16 μM Aprotinin, 0.01 mM Bestatin, 3 μM E-64, 4 μM Leupeptin, and 2 μM Pepstatin) on ice for 30′. Cells were then homogenized with a dounce homogenizer on ice. The lysates were cleared by centrifugation. One ml of the cleared lysate was incubated with 5 μg of various small G proteins, expressed as GST fusion proteins and bound to glutathione-agarose beads, at 4° C. for 2 hrs. The protein-agarose complex was pelleted by brief centrifugation and washed for 4 times 5′ each with the lysis buffer at room temperature. The complex was then resuspended in SDS sample buffer, boiled and run on a SDS-PAGE and Western blotted with an anti-H19G5 monoclonal antibody. As shown in FIG. 12, H19G5 was found to bind to Cdc42. No binding was detected with Rac1 or RhoA. Lane 8 is His-tagged Cdc42 and in lane 9 His-tagged Cdc42-agarose complex was boiled for 5′ before adding to the H19G5 cell lysate. This result showed that H19G5 specifically interacts with Cdc42 protein and there is no non-specific interaction with agarose beads. The results presented herein suggest that 19G5 protein may play an important role, by virtue of regulating a small GTPase such as Cdc42Hs, in a variety of cellular activities. For example, Cdc42 has been shown to regulate actin polymerization and focal adhesion complex formation which in turn is necessary for filopedia formation (Nobes and Hall, Cell 81: 53-62[1995]). Cdc42 and rac have also been shown to regulate Jun N-terminal kinase (JNK) activity via the MAP kinase pathway (Coso et al., Cell 81: 1137-1146[1995]; Minden et al., Cell 81: 1147-1157[1995]; Olson et al., Science 269: 1270-1272 [1995]), an evolutionarily conserved and ubiquitous signal transduction pathway that impacts upon a number of important cellular functions.

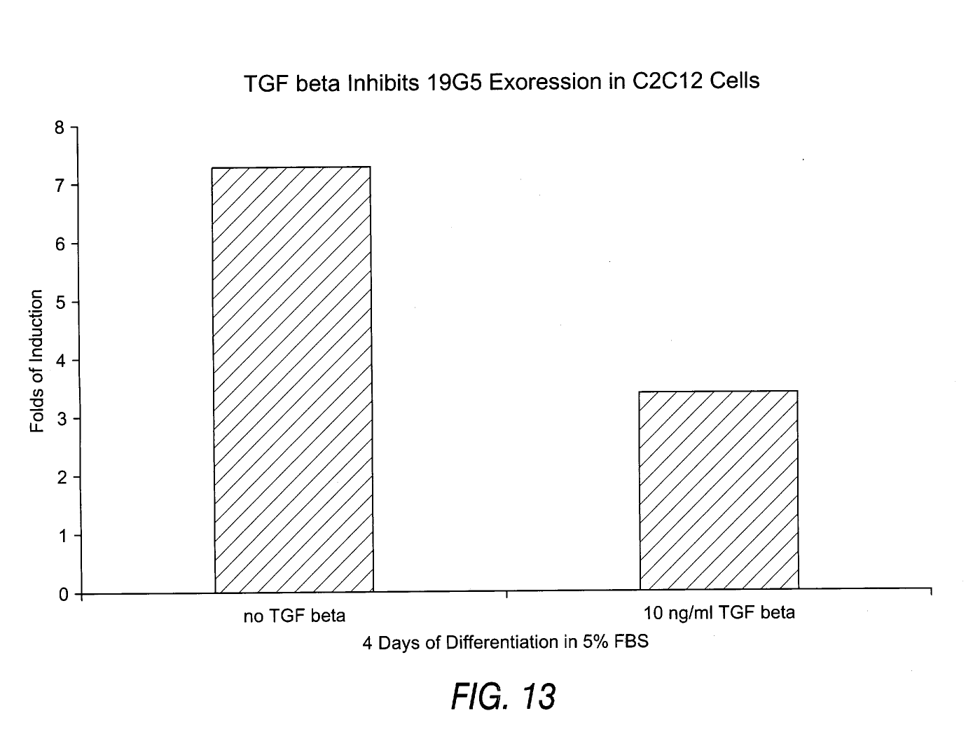

C2C12 myoblasts cells (ATCC Catalog No. CRL-1772) can be induced to differentiate into myotubes when placed in a medium containing 2% horse serum (Lechner et al., Proc. Natl. Acad. Sci. USA 93: 4355-4359[1996]). FIG. 13 shows phase contrast micrographs of C2C12 myoblasts undergoing differentiation into myotubes. At Day 0, undifferentiated C2C12 cells with typical myoblast morphology can be seen. Once induced to differentiate, as shown here at Day 3 and 5 after induction, an increasingly larger number of cells with typical morphology of differentiated myotubes, i.e. large, elongated, multinucleated syncytial cells, could be seen. The induction of Myogenin expression was monitored during differentiation. Myogenin is not expressed in myoblasts, however, its expression is strongly induced when myoblasts undergo differentiation into myotubes. Thus, it acts as a biochemical marker of myotubes. FIG. 14 shows induction of myogenin protein expression when C2C12 myoblast cells are induced to differentiate into myotubes. C2C12 cells were cultured and induced to differentiate by placing in a medium containing 2% horse serum. Cells were lysed in radioimmunoprecipitation (RIPA) buffer (1X PBS containing 1% Igepal CA-630, 0.5% sodium deoxycholate and 0.1% SDS) on ice for 30′. Total lysates were cleared by centrifugation at 10,000 rpm for 10′. Protein concentration of each lysate was measure using the BCA method. SDS sample buffer was added to the total lysate and boiled for 3′. Equal amount of total protein of each sample was run on SDS-PAGE and blotted using an anti-myogenin monoclonal antibody. The result demonstrated that Myogenin expression was induced concomitantly with differentiation of C2C12 myoblasts into myotubes under the conditions used for induction.

The expression of 19G5 RNA was also monitored at various stages during differentiation of myoblasts into myotubes. Undifferentiated C2C12 cells were plated on 6 cm dishes at 2.4×10⁴ cells/cm² and cultured in growth medium with 10% fetal bovine serum for 24 hrs to about confluence. Cells were washed with PBS and induced to differentiate into myotubes in differentiation medium with 2% horse serum. Total RNA was isolated from cells at 1, 2, or 4 days post-induction using Qiagen's Rneasy kit according to the manufacture's instruction. The expression of 19G5 transcript was analyzed using Taqman assay. One microgram of total RNA, isolated at various time points post-induction, was reverse transcribed into cDNA using PE Biosystems Reagents and Multiscribe enzyme according to manufacture's instruction. Ten ng of cDNA was added to 1× master mix, and the primers and probe for the gene of interest were added according to manufacture's instructions. The reaction were carried out in the ABI Prism 7700 Detection System. The quantity of 19G5 and the quantity for 18S were determined for each sample, and the ratio of 19G5/18S was used to evaluate differences in the level of 19G5 expression in various samples. Comparison of the values thus obtained with pre-induction values allowed to determine fold induction of 19G5 expression during differentiation. As shown in FIG. 15, expression of 19G5 transcript was significantly increased during differentiation of C2C12 myoblasts into myotubes. The level of induction reached to about 10-fold at 4 days post-induction. Increased expression of 19G5 likely reflects a specific function in myotubes.

TGF-β is known to inhibit differentiation of C2C12 myoblast into myotubes (Katagiri et al., J. Cell Biol. 127: 1755-1766[1994]; Namiki et al., J. Biol. Chem. 272: 22046-22052[1997]). Therefore, the effect of TGF-β on the induction of expression of 19G5 during differentiation of C2C12 cells was examined. C2C12 cells were plated in 6 cm dishes at 2.4×10⁴ cells/cm² and cultured in the growth medium for 24 hours. Cells were then rinsed with PBS and induced to differentiate for 4 days in the medium containing 5% fetal bovine serum either in the absence or in the presence of 10 ng/ml of TGF-β. Total RNA was isolated from cells using Qiagen's Rneasy kit. Induction of 19G5 expression in TGF-β treated or untreated cells over undifferentiated C2C12 cells was measured by Taqman assay. As shown in FIG. 16, the induction of 19G5 expression during C2C12 differentiation is inhibited by TFG-β. TGF-β is a powerful regulator of cell growth and differentiation and regulation of expression of 19G5 by TGF-β likely represents an important physiological event with significant relevance to normal and abnormal changes in cardiac cells. Furthermore, the results suggest a possible inolvement of 19G5 in myogenesis.

### EXAMPLE 2

### Polypeptides which can Mediate Signal Transduction

The polypeptides of the present invention, such as the specific embodiment shown in SEQ ID NOs: 1, 4, 6, 7, 8 or 9 may be prepared by any known techniques. Conveniently, the polypeptides may be prepared using the solid-phase synthetic technique initially described by Merrifield in J. Am. Chem. Soc. 15:2149-2154 (1963). Other peptide synthesis techniques may be found, for example, in M. Bodanszky et al., PEPTIDE SYNTHESIS, John Wiley & Sons, 2d Ed. (1976) as well as in other reference works known to those skilled in the art. A summary of peptide synthesis techniques may be found in J. Stuart and J. D. Young, SOLID PHASE PEPTIDE SYNTHELIA, Pierce Chemical Co., Rockford, Ill. (1984). The synthesis of peptides by solution methods may also be used, as described in THE PROTEINS, Vol-II, 3d Ed., Neurath, H. et al., Eds., p.105-237, Academic Press, New York, N.Y. (1976). Appropriate protective groups for use in such syntheses will be found in the above texts as well as in J. F. W. McOmie, PROTECTIVE GROUPS IN ORGANIC CHEMISTRY, Plenum Press, New York, N.Y. (1973). In general, these synthetic methods involve the sequential addition of one or more amino acid residues or suitably protected amino acid residues to a growing peptide chain. Normally, either the amino or carboxyl group of the first amino acid residue is protected by a suitable, selectively-removable protecting group. A different, selectively-removable protecting group is utilized for amino acids containing a reactive side group, such as lysine.

Using a solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The polypeptides of the invention preferably are devoid of benzylated or methylbenzylated amino acids. Such protecting group moieties may be used in the course of synthesis, but they are removed before the polypeptides are used. Additional reactions may be necessary, as described elsewhere to form intramolecular linkages to restrain conformation, if desired. The polypeptides of the present invention may also be linked to an additional sequence of amino acids either or both at the N-terminus and at the C-terminus. Such additional amino acid sequences, or linker sequences, can be conveniently affixed to a detectable label, solid matrix, or carrier. Typical amino acid residues used for linking are tyrosine, cysteine, lysine, glutamic acid and aspartic acid, or the like.

Of course, the present polypeptides may also be prepared by recombinant DNA techniques as described, for example, in Sambrook, et al., MOLECULAR CLONING: A LABORATORY MANUAL, Chapters 1-18, Second Edition (Cold Spring Harbor N.Y. 1989), and as detailed in Examples 3-4 infra. The present invention also relates to vectors comprising DNA molecules of the present invention, host cells which are genetically engineered with vectors of the invention and the production of polypeptides of the invention by recombinant techniques. Host cells may be genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying genes. The culture conditions, such as temperature, pH and the like, are preferably those previously used with the host cell selected for expression, and will be apparent to the skilled artisan.

### EXAMPLE 3

### Hosts, Vectors and DNA Encoding Polypeptides which can Mediate Signal Transduction

The DNA molecules of the present invention may be employed for producing the polypeptides of the present invention by recombinant techniques. More particularly, the present invention also includes recombinant constructs comprising one or more of the sequences broadly described above. Thus, for example, the DNA molecule sequence may be included in any one of a variety of expression vehicles, in particular vectors or plasmids for expressing such a polypeptide. Such vectors include chromosomal, nonchromosomal and synthetic DNA sequences, e.g., derivatives of SV40; bacterial plasmids; phage DNA; yeast plasmids; vectors derived from combinations of plasmids and phage DNA; viral DNA such as vaccinia, adenovirus, fowl pox virus, and pseudorabies. The following vectors are provided by way of example. Bacterial: pQE70, pQE60, pQE-9 (Qiagen), pBS, phagescript, psiX174, pBluescript SK, pBsKS, pNH8a, pNH16a, pNH18a, pNH46a (Stratagene); pTRC99A, pKK223-3, pKK233-3, pDR540, PRIT5 (Pharmacia). Eukaryotic: pWLneo, pSV2cat, pOG44, pXT1, pSG (Stratagene); pSVK3, pBPV, pMSG, PSVL (Pharmacia). However, any other vector or plasmid may be used as long as they are replicable and viable in the host. The vector containing the appropriate DNA sequence, as well as an appropriate promoter or control sequence, may be employed to transform an appropriate host to permit the host to express the polypeptides of the present invention. Representative examples of appropriate hosts include: bacterial cells, such as E. coli, Salmonella typhimurium, Streptomyces; fungal cells, such as yeast; insect cells, such as Drosophila S2 and Spodoptera Sf9; animal cells, such as CHO, COS or Bowes melanoma; adenoviruses; plant cells, etc. The selection of an appropriate host is deemed to be within the scope of those skilled in the art.

The appropriate DNA sequence may be inserted into the vector by a variety of procedures. In general, the DNA sequence is inserted into an appropriate restriction endonuclease site by procedures known in the art. Such procedures and others are deemed to be within the scope of those skilled in the art.

The DNA sequence in the expression vector may be operatively linked to an appropriate expression control sequence(s) (promoter) to direct mRNA synthesis. Suitable promoter regions can be selected from any desired gene using CAT (chloramphenicol transferase) vectors or other vectors with selectable markers. Two appropriate vectors are pKK232-8 and pCM7. Particular named bacterial promoters include laci, lacZ, T3, T7, gpt, lambda P_{R}, P_{L}and trp. Eukaryotic promoters include CMV immediate early, HSV thymidine kinase, early and late SV40, LTRs from retrovirus, and mouse metallothionein-I. Selection of the appropriate vector and promoter is well within the level of skill in the art. The expression vector may also contain a ribosome binding site for translation initiation and a transcription terminator. The vector may also include appropriate sequences for amplifying expression.

In addition, the expression vectors preferably may contain a gene to provide a phenotypic trait for selection of transformed host cells such as dihydrofolate reductase or neomycin resistance for eukaryotic cell culture, or such as tetracycline or ampicillin resistance in E. coli.

An embodiment of the invention is an isolated DNA molecule comprising the nucleotide sequence of SEQ ID NOs: 2, 3 or 5. This nucleotide sequence, or fragments or functional equivalents thereof, may be used to generate recombinant DNA molecules that direct the expression of the polypeptides of the present invention, or functionally active peptides or functional equivalents thereof, in appropriate host cells. Due to the degeneracy of the nucleotide coding sequence, other DNA sequences which encode substantially the same amino acid sequences as depicted in SEQ ID NOS: 1, 4, 6, 7, 8 or 9, or analogs or fragments thereof, may be used in the practice of the invention for the cloning and expression of a mediator of signal transduction. Such alterations include deletions, additions or substitutions of different nucleotide residues resulting in a sequence that encodes the same or a functionally equivalent gene product. The gene product may contain deletions, additions or substitutions of amino acid residues within the sequence, which result in a silent change thus producing a bioactive product. Such amino acid substitutions may be made on the basis of similarity in polarity, charge, solubility, hydrophobicity, hydrophilicity, the amphipathic nature of the residues involved and/or on the basis of crystallographic data. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine.

Techniques well known to those skilled in the art for the isolation of DNA, generation of appropriate restriction fragments, construction of clones and libraries, and screening recombinants may be used. For a review of such techniques, see, for example, Sambrook, et al., supra, the disclosure of which is hereby incorporated by reference. Also, the 5′ untranslated and coding regions of the nucleotide sequence could be altered to improve the translational efficiency of the mRNA. In addition, based on X-ray crystallographic data, sequence alterations could be undertaken to improve protein stability, e.g., introducing disulfide bridges at the appropriate positions, and/or deleting or replacing amino acids that are predicted to cause protein instability. These are only examples of modifications that can be engineered to produce a more active or stable protein, more protein, or even change the substrate specificity of the protein.

### EXAMPLE 4

### Cells Transformed with Recombinant Vectors Containing DNA Encoding Polypeptides which can Mediate Signal Transduction

In a further embodiment, the present invention relates to host cells containing the above-described construct. The host cell can be a higher eukaryotic cell, such as a mammalian cell, or a lower eukaryotic cell, such as a yeast cell, or the host cell can be a prokaryotic cell, such as a bacterial cell. The host cell preferably may secrete the recombinant protein. Introduction of the construct into the host cell can be effected by calcium phosphate transfection, DEAE-Dextran mediated transfection, or electroporation (L. Davis et al., BASIC METHODS IN MOLECULAR BIOLOGY, 1986)).

The constructs in host cells can be used in a conventional manner to produce the gene product encoded by the recombinant sequence. Alternatively, the polypeptides of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., supra.

Transcription of a DNA encoding the polypeptides of the present invention by higher eukaryotes may be increased by inserting an enhancer sequence into the vector. Enhancers are cis-acting elements of DNA, usually from about 10 to 300 bp, that act on a promoter to increase its transcription. Examples include the SV40 enhancer on the late side of the replication origin (base pair 100 to 270), a cytomegalovirus early promoter enhancer, a polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is preferably assembled in appropriate phase with translation, initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use may be constructed by inserting a structural DNA sequence encoding a desired protein together with suitable translation, initiation and termination signals in operable reading phase with a functional promoter. The vector may comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis.). These pBR322 backbone sections are combined with an appropriate promoter and the structural sequence to be expressed.

Following transformation of a suitable host strain and growth of the host strain to an appropriate cell density, the selected promoter may be de-repressed by appropriate means (e.g., temperature shift or chemical induction) and cells may be cultured for an additional period. Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents. Various mammalian cell culture systems can also be employed to express recombinant polypeptides. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors may comprise an origin of replication, a suitable promoter and enhancer, and also any necessary ribosome binding sites, polyadenylation sites, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements.

The polypeptides of the present invention may be recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic-procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eukaryotic carbohydrates or may be non-glycosylated. Polypeptides of the invention may also include an initial methionine amino acid residue.

In particular, two baculovirus expression constructs of a wild-type and a mutant H19G5 C-terminal kinase domain have been constructed. The wild-type kinase domain construct was made by cloning a CDNA fragment which encodes the amino acid residues 1002 to 1314 of the H19G5 contig protein sequence into the pFastBac HTc vector. The mutant kinase domain construct contains the same amino acid sequence as the wild-type kinase domain except that Tyr residue at position 1213 was changed to a Glu in an attempt to create a constitutively active kinase. Both recombinant proteins contain a his-tag at the N-terminus.

### EXAMPLE 5

### Pharmaceutically Acceptable Salts of Polypeptides which can Mediate Signal Transduction

Any peptide of the present invention may be used in the form of a pharmaceutically acceptable salt. Suitable acids which are capable of forming salts with the peptides of the present invention include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanolamines (e.g., ethanolamine, diethanolamine and the like).

### EXAMPLE 6

### Pharmaceutical Compositions Containing Polypeptides which can Mediate Signal Transduction

For use in a method of identification, prevention or treatment, such as the identification, prevention or treatment of infection of a mammalian host by a microorganism, the polypeptides of the present invention may be present in a pharmaceutical composition in admixture with a pharmaceutically acceptable sterile vehicle. The pharmaceutical composition may be compounded according to conventional pharmaceutical formulation techniques.

The vehicle may take a wide variety of forms depending on the form of preparation desired for administration, e.g., sublingual, rectal, nasal, oral or parenteral. Compositions for oral dosage form may include any of the usual pharmaceutical media, such as, for example, water, oils, alcohols, flavoring agents, preservatives, coloring agents and the like in the case of oral liquid preparations (e.g., suspensions, elixirs and solutions) or carriers such as starches, sugars, diluents, granulating agents, lubricants, binders, disintegrating agents and the like in the case of oral solid preparations (e.g., powders, capsules and tablets). Controlled release forms may also be used. Because of their ease in administration, tablets and capsules represent an advantageous oral dosage unit form, in which case solid pharmaceutical carriers may be employed. If desired, tablets may be sugar coated or enteric coated by standard techniques.

For compositions to be administered parenterally, the carrier will usually comprise sterile water, although other ingredients to aid solubility or for preservation purposes may be included. Injectable suspensions may also be prepared, in which case appropriate liquid carriers, suspending agents and the like may be employed. The parenteral routes of administration may be intravenous injection, intramuscular injection or subcutaneous injection.

For intravenous administration, the polypeptides may be dissolved in an appropriate intravenous delivery vehicle containing physiologically compatible substances such as sodium chloride, glycine and the like, having a buffered pH compatible with physiologic conditions. Such intravenous delivery vehicles are known to those skilled in the art.

The polypeptides of the invention may be administered to subjects where mediation of signal transduction is desired. The peptides may be administered by any convenient means that will result in the delivery to the subject of an effective amount to mediate signal transduction. Oral administration is presently contemplated as a preferred administration route. The amount administered will depend on the activity of the particular compound administered, which may readily be determined by those of ordinary skill in the art.

### EXAMPLE 7

### Monoclonal Antibodies Against Polypeptides which can Mediate Signal Transduction

Another embodiment of the present invention relates to a monoclonal antibody to the polypeptides of the present invention (or an antigenic portion thereof), which may be produced by methods recognized in the art, including the formation of monoclonal antibody-producing hybridomas (Kohler, G., and C. Milstein, Nature 256:495-497 (1975); Eur. J. Immunol. 6:511-519 (1976)). By fusing antibody-forming cells (spleen lymphocytes) with myeloma cells (malignant cells of bone marrow primary tumors), a hybrid cell line is created from a single fused cell hybrid (called a hybridoma or clone) having certain inherited characteristics of both the lymphocytes and myeloma cell lines. Like the lymphocytes (taken from animals primed with sheep red blood cells as antigen), the hybridomas secreted a single type of immunoglobulin specific to the antigen; moreover, like the myeloma cells, the hybrid cells had the potential for indefinite cell division. The combination of these two features offered distinct advantages over conventional antisera. Whereas antisera derived from vaccinated animals are variable mixtures of polyclonal antibodies which never can be reproduced identically, monoclonal antibodies are highly specific immunoglobulins of a single type. The single type of immunoglobulin secreted by a hybridoma is specific to one and only one antigenic determinant, or epitope, on the antigen, a complex molecule having a multiplicity of antigenic determinants. For instance, if the antigen is a protein, an antigenic determinant may be one of the many peptide sequences (generally 6-7 amino acids in length (Atassi, M. Z., Molec. Cell. Biochem. 32:21-43 (1980)) within the entire protein molecule. Hence, monoclonal antibodies raised against a single antigen may be distinct from each other depending on the determinant that induced their formation; but for any given clone, all of the antibodies it produces are identical. Furthermore, the hybridoma cell line can be reproduced indefinitely, is easily propagated in vitro or in vivo, and yields monoclonal antibodies in extremely high concentration.

### EXAMPLE 8

### Therapeutic Monoclonal Antibodies Against Polypeptides which can Mediate Signal Transduction

The monoclonal antibodies of the present invention can have potential immunotherapeutic value (Oldham, R. K., J. Clin. Oncol., 1:582-590 (1983); Miller, R. A. et al., Blood, 62:988-995 (1983); Miller R. A. et al., New Engl. J. Med. 306:517-522 (1982); Ritz, J. and Schlossman, S., Blood, 59:1-11 (1982); and Kirch, M. E. and Ulrich, H., J. Immunol. 127:805-810 (1981) (investigating the therapeutic efficacy in both animal and human subjects)). In addition, the monoclonal antibodies can be used in cytotoxic drug-antibody conjugates similar to those described in Beverly, P. C. L., Nature, 297:358-9 (1982); Krolick, K. A. et al., Nature, 295:604-5 (1982); Krolick, K. A. et al., Proc. Natl. Acad. Sci. U.S.A., 77:5419-23 (1980); Arnon, R. and Sela, M., Immunol. Rev., 62:5-27 (1982); Raso, V. et al., Cancer Res., 42:457-64 (1982); and DeWeger, R. A. and Dullens, H. F. J., Immunol. Rev. 62:29-45 (1982).

In an embodiment of the invention, purified polypeptides of the present invention (or an antigenic portion thereof) can be used as an antigen or immunogen. In addition, microorganisms expressing H19G5 protein or polypeptide fragments thereof also represent potential antigens or sources of antigen with which to immunize animals to obtain somatic cells for fusion. Somatic cells with the potential for producing antibody and, in particular, B lymphocytes, are suitable for fusion with a B-cell myeloma line. Those antibody-producing cells that are in the dividing plasmablast stage fuse preferentially. Somatic cells may be derived from the lymph nodes, spleens and peripheral blood of primed animals and the lymphatic cells of choice depending to a large extent on their empirical usefulness in the particular fusion system. Once-primed or hyperimmunized animals can be used as a source of antibody-producing lymphocytes. Mouse lymphocytes give a higher percentage of stable fusions with mouse myeloma lines. However, the use of rat, rabbit, and frog cells is also possible. Alternatively, human somatic cells capable of producing antibody, specifically B lymphocytes, are suitable for fusion with myeloma cell lines. While B lymphocytes from biopsied spleens or lymph nodes of individual may be used, the more easily accessible peripheral blood B lymphocytes are preferred. The lymphocytes may be derived from patients with diagnosed carcinomas.

Specialized myeloma cell lines have been developed from lymphocyte tumors for use in hybridoma-producing fusion procedures (Kohler, G., and C. Milstein, Eur. J. Immunol. 6:511-519 (1976); M. Schulman et al., Nature 276: 269-270 (1978)). Examples of mycloma cell lines that may be used for the production of fused cell hybrids include X63-Ag8, NSI-Ag4/1, MPC11-45.6TG1.7, C63-Ag8.653, Sp2/0-Ag14, FO, and S194/5XX0.BU.1, all derived from mice; 210.RCY3. Ag1.2.3, U-226AR, and GM1500GTGAL2, all derived from rats; and U-226AR and GM1500GTGAL2, derived from humans, (G. J. Hammerling, U. Hammerling, and J. F. Kearney (editors), Monoclonal Antibodies and T-cell Hybridomas in: J. L. Turk (editor) RESEARCH MONOGRAPHS IN IMMUNOLOGY, Vol. 3, Elsevier/North Holland Biomedical Press, NY (1981)).

Methods for generating hybrids of antibody-producing spleen or lymph node cells and myeloma cells usually comprise mixing somatic cells with myeloma cells in a 2:1 proportion (though the proportion may vary from about 20:1 to about 1:1), respectively, in the presence of an agent or agents (chemical or electrical) that promote the fusion of cell membranes. It is often preferred that the same species of animal serve as the source of the somatic and myeloma cells used in the fusion procedure. Fusion methods have been described by Kohler and Milstein (Nature 256:495-497 (1975) and Eur. J. Immunol. 6:511-519 (1976), and by Gefter et al. (Somatic Cell Genet. 3:231-236 (1977)). The fusion-promotion agents used by those investigators were Sendai virus and polyethylene glycol (PEG), respectively.

Once the desired fused cell hybrids have been selected and cloned into individual antibody-producing cell lines, each cell line may be propagated in either of two standard ways. A sample of the hybridoma can be injected into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can be tapped to provide monoclonal antibodies in high concentration. Alternatively, the individual cell lines may be propagated in vitro in laboratory culture vessels; the culture medium, also containing high concentrations of a single specific monoclonal antibody, can be harvested by decantation, filtration or centrifugation.

### EXAMPLE 9

### Diagnostic Monoclonal Antibodies Against Polypeptides which can Mediate Signal Transduction

The monoclonal antibodies of this invention can be used as probes in detecting discrete antigens expressed by tissue or cell samples. The expression or lack of expression of these antigens can provide clinically exploitable information that is not apparent after standard histopathological evaluations. It may thus be possible to correlate the immuno-phenotypes of individual tissue or cell samples with various aspects of signal transduction and responsiveness to certain types of therapies, thereby establishing important classifications of prognosis.

The use of the monoclonal antibodies described herein can be extended to the screening of human biological fluids for the presence of the specific antigenic determinant recognized. In vitro immunoserological evaluation of sera withdrawn from patients thereby permits non-invasive diagnosis of disease. By way of illustration, human fluids, such as pleural fluids or lymph, can be taken from a patient and assayed for the specific epitope, either as released antigen or membrane-bound on cells in the sample fluid, using monoclonal antibodies against the polypeptides of the present invention in standard radioimmunoassays or enzyme-linked immunoassays known in the art or competitive binding enzyme-linked immunoassays.

The monoclonal antibodies of this invention are potentially useful for targeting diseased tissue or cells in vivo. They can therefore be used in humans for localization and monitoring of the microbial infection. For this application, it is preferable to use purified monoclonal antibodies. Purification of monoclonal antibodies for human administration by ammonium sulfate or sodium sulfate precipitation followed by dialysis against saline and filtration sterilization has been described by Miller et al. (in: HYBRIDOMAS IN CANCER DIAGNOSIS AND THERAPY, (1982), p. 134).

Alternatively, immunoaffinity chromatography techniques may be used to purify the monoclonal antibodies. The purified monoclonal antibodies can be labeled with radioactive compounds, for instance, radioactive iodine, and administered to a patient intravenously. After localization of the antibodies at the infection site, they can be detected by emission tomographical and radionuclear scanning techniques, thereby pinpointing the location of the infection. Experimental radioimmunodetection with monoclonal antibodies may occur by external scintigraphy.

Passive monoclonal serotherapy may be a potential use for the monoclonal antibodies of this invention. By way of illustration, purified anti-H19G5 monoclonal antibody is dissolved in an appropriate carrier, e.g., saline, with or without human albumin, at an appropriate dosage and is administered to a patient. The monoclonal antibodies are preferably administered intravenously, e.g., by continuous intravenous infusion over several hours, as in Miller et al, supra. Infusions can be administered over a period of weeks during which the anti-microbial effects are monitored.

### EXAMPLE 10

### Anti-Idiotypic Antibodies to Antibodies Against Polypeptides which can Mediate Signal Transduction

In an alternate embodiment, the antibodies described herein are used to stimulate the production of corresponding anti-idiotypic antibodies. In brief, anti-idiotypic antibodies, or antiidiotypes are antibodies directed against the antigen combining region or variable region (idiotype) of another antibody. Based on Jerne's network model of idiotypic relationships (Jerne, Ann. Immunol. 125:373 (1974); Jerne et al., EMBO 1:234 (1982)), immunization with an antibody molecule expressing a paratope (antigen-combining site) for a given antigen should produce a group of anti-antibodies, some of which share with the antigen a complementary structure to the paratope. Immunization with a subpopulation of antiidiotypic antibodies should in turn produce a subpopulation of antiidiotypic antibodies which bind the initial antigen. Thus, the administration of the monoclonal antibodies of the present invention may result in a modification of the host's immune response, as the consequence of the formation of anti-idiotypic antibodies which may develop during therapy with the monoclonals.

### EXAMPLE 11

### Monoclonal Antibody-Drug Conjugates

The monoclonal antibodies of this invention can be used in conjunction with a broad spectrum of pharmaceutical or cytotoxic agents that selectively affect diseased tissue or cells over normal tissues or cells in the mammalian host. The methods used for binding the cytotoxic agents to the monoclonal antibody molecule can involve either non-covalent or covalent linkages. Since non-covalent bonds are more likely to be broken before the antibody complex reaches the target site, covalent linkages are preferred. For instance, carbodiimide can be used to link carboxy groups of the pharmaceutical agent to amino groups of the antibody molecule. Bifunctional agents such as dialdehydes or imidoesters can be used to link the amino group of a drug to amino groups of the antibody molecule. The Schiff base reaction can be used to link drugs to antibody molecules. This method involves the periodate oxidation of a drug or cytotoxic agent that contains a glycol or hydroxy group, thus forming an aldehyde that is then reacted with the antibody molecule. Attachment occurs via formation of a Schiff base with amino groups of the antibody molecule. Additionally, drugs with reactive sulfhydryl groups have been coupled to antibody molecules.

### EXAMPLE 12

### Diagnostic Kit

Another embodiment of the invention relates to a diagnostic kit for detecting diseased tissue or cells using an antibody against a polypeptide which can mediate signal transduction. The diagnostic kit may further comprise, where necessary, other components of the signal producing system, including agents for reducing background interference, control reagents, or an apparatus, container or other solid support for conducting the test. The binding of antibody to the target can be detected by well known methods, including radiation (e.g., use of a radioactive nucleotide), colorimetry (e.g., use of an enzyme that can cause a color change in a substrate), fluorescence (e.g., use of a dye such as propidium iodide, fluorescein, or rhodamine), and luminescence (e.g., use of an alkaline phosphatase substrate that releases photons upon cleavage or luciferin). Detection can be qualitative or quantitative.

### EXAMPLE 13

### Gene Therapy

Another embodiment of the present invention involves the use of the DNA of the present invention in gene therapy applications. Gene therapy has been broadly defined as “the correction of a disease phenotype through the introduction of new genetic information into the affected organism” (Roemer, K. and Friedmann, T., Eur. J. Biochem. 208: 211-225 (1992)). Two basic approaches to gene therapy have evolved: (1) ex vivo gene therapy and (2) in vivo gene therapy. In ex vivo gene therapy, cells are removed from a subject and cultured in vitro. A functional replacement gene is introduced into the cells (transfection) in vitro, the modified cells are expanded in culture, and then reimplanted in the subject. These genetically modified, reimplanted cells are reported to secrete detectable levels of the transfected gene product in situ (Miller, A. D., Blood 76: 271-278 (1990)) and Selden, R. F., et al., New Eng. J. Med. 317: 1067-1076 (1987)). The development of improved retroviral gene transfer methods (transduction) facilitates the transfer into and subsequent expression of genetic material by somatic cells (Cepko, C. L., et al., Cell 37: 1053-1062 (1984)). Accordingly, retrovirus-mediated gene transfer has been used in clinical trials to mark autologous cells and as a way of treating genetic disease (Rosenberg, S. A., et al., New Eng. J. Med. 323: 570-578 (1990); Anderson, W. F., Human Gene Therapy 2: 99-100 (1991)). Several ex vivo gene therapy studies in humans are reported (reviewed in Anderson, W. F., Science 256: 808-813 (1992) and Miller A. D., Nature 357: 455-460 (1992)).

In in vivo gene therapy, target cells are not removed from the subject. Rather, the transferred gene is introduced into cells of the recipient organism in situ, that is, within the recipient. In vivo gene therapy has been examined in several animal models (reviewed in Felgner, P. L. and Rhodes, G., Nature 349: 351-352 (1991)). Publications have reported the feasibility of direct gene transfer in situ into organs and tissues such as muscle (Ferry, N., et al., Proc. Natl. Acad. Sci. 88: 8377-8781 (1991); Quantin, G., et al., Proc. Natl. Acad. Sci. USA 89: 2581-2584 (1992)), hematopoietic stem cells (Clapp, D. W., et al., Blood 78: 1132-1139 (1991)), the arterial wall (Nabel, E. G., et al., Science 244: 1342-1344 (1989)), the nervous system (Price, J. D., et al., Proc. Natl. Acad. Sci. 84: 156-160 (1987)), and lung (Rosenfeld, M. A., et al., Science 252: 431-434 (1991)). Direct injection of DNA into skeletal muscle (Wolff, J. A., et al., Science 247: 1465-1468 (1990)), heart muscle (Kitsis, R. N., et al., Proc. Natl. Acad. Sci. USA 88: 4138-4142 (1991)) and injection of DNA-lipid complexes into the vasculature (Lim, C. S., et al., Circulation 83: 2007-2011 (1991); Ledere, G. D., et al., J. Clin. Invest. 90: 936-944 (1992); Chapman, G. D., et al., Circ. Res. 71: 27-33 (1992)) also have been reported to yield a detectable expression level of the inserted gene product(s) in vivo.

Recent gene therapy efforts have been aimed at the identification of various cell types for transformation, including keratinocytes (Morgan, J. R., et al., Science 237: 1476-1479 (1987)), fibroblasts (Palmer, T. D., et al., Proc. Natl. Acad. Sci. 88: 1330-1334 (1991); Garver Jr., R. I., et al., Science 237: 762-764 (1987); International Patent Application PCT/US92/01890, having publication number WO 92/15676), lymphocytes (Reimann, J. K., et al., J. Immunol. Methods 89: 93-101 (1986)), myoblasts (Barr, E. and Leiden, J. M., Science 254: 1507-1509 (1991); Dai, Y. et al., PNAS 89: 10892-10895 (1992); Roman, M., et al., Somatic Cell and Molecular Genetics 18: 247-258 (1992)), smooth muscle cells (Lynch, C. M. et al., Proc. Natl. Acad. Sci. USA 89: 1138-1142 (1992)), and epithelial cells (Nabel, E. G., et al., Science 244: 1342-1344 (1989)), International Patent Application PCT/US89/05575 (having publication number WO 90/06997), the contents of which references and patent/patent applications are incorporated herein by reference.

The delivery of an effective dose of a prophylactic or therapeutic agent in situ depends on the efficiency of transfection (or transduction) as well as the number of target cells. Epithelial cell-based gene therapy, in particular, involves a relatively small area available in situ for receiving genetically modified epithelial cells. The delivery of an effective dose of prophylactic or therapeutic agent in situ thus depends upon the total number of implanted epithelial cells.

In one embodiment of the invention, exogenous genetic material (e.g., a cDNA encoding a polypeptide of the present invention) is introduced into a syngeneic host cell ex vivo or in vivo by genetic transfer methods, such as transfection or transduction, to provide a genetically modified host cell. Various expression vectors (i.e., vehicles for facilitating delivery of exogenous genetic material into a target cell) are known to one skilled in the art.

Transfection refers to the insertion of nucleic acid into a mammalian host cell using physical or chemical methods. Several transfection techniques are known to those of ordinary skill in the art including: calcium phosphate DNA co-precipitation (METHODS IN MOLECULAR BIOLOGY, Vol. 7, Gene Transfer and Expression Protocols, Ed. E. J. Murray, Humana Press (1991)); DEAE-dextran; electroporation; cationic liposome-mediated transfection; and tungsten particle-facilitated microparticle bombardment (Johnston, S. A., Nature 346: 776-777 (1990)). Strontium phosphate DNA co-precipitation (Brash D. E. et al. Molec. Cell. Biol. 7: 2031-2034 (1987)) is a preferred transfection method.

In contrast, transduction refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced host cell. A host cell that has been transduced with a chimeric DNA virus (e.g., an adenovirus carrying a cDNA encoding a therapeutic agent) will not have the exogenous genetic material incorporated into its genome, but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an enhancer is simply any non-translated DNA sequence which works contiguous with the coding sequence (in cis) to change the basal transcription level dictated by the promoter. Preferably, the exogenous genetic material is introduced into the host cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or housekeeping functions: hypoxanthine phosphoribosyl transferase (HPRT), dihydrofolate reductase (DHFR) (Scharfinann et al., Proc. Natl. Acad. Sci. USA 88: 4626-4630 (1991)), adenosine deaminase, phosphoglycerol kinase (PGK), pyruvate kinase, phosphoglycerol mutase, the beta-actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40, the long terminal repeats (LTRs) of Moloney Leukemia Virus and other retroviruses, and the thymidine kinase promoter of Herpes Simplex Virus, among many others. Accordingly, any such constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response, and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified host cell. If the gene encoding the prophylactic or therapeutic agent is under the control of an inducible promoter, delivery of the agent in situ is triggered by exposing the genetically modified cell in situ to conditions for permitting transcription of the prophylactic or therapeutic agent, e.g., by intraperitoneal injection of specific inducers of the inducible promoters which control transcription of the agent. For example, in situ expression by genetically modified host cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene (i.e., whether the promoter is constitutive or inducible, strong or weak); (2) the number of copies of the exogenous gene that are inserted into the host cell; (3) the number of transduced/transfected host cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the prophylactic or therapeutic agent by the genetically modified host cell. Selection and optimization of these factors for delivery of an effective dose of a particular prophylactic or therapeutic agent is deemed to be within the scope of one of skill in the art, taking into account the above-disclosed factors and the clinical profile of the patient.

In addition to at least one promoter and at least one heterologous nucleic acid encoding the prophylactic or therapeutic agent, the expression vector preferably includes a selection gene, for example, a neomycin resistance gene, for facilitating selection of host cells that have been transfected or transduced with the expression vector. Alternatively, the host cells are transfected with two or more expression vectors, at least one vector containing the gene(s) encoding the prophylactic or therapeutic agent(s), the other vector containing a selection gene. The selection of a suitable promoter, enhancer, selection gene and/or signal sequence is deemed to be within the scope of one skilled in the art.

The prophylactic or therapeutic agent can be targeted for delivery to an extracellular, intracellular or membrane location. If it is desirable for the gene product to be secreted from the host cells, the expression vector is designed to include an appropriate secretion signal sequence for secreting the therapeutic gene product from the cell to the extracellular milieu. If it is desirable for the gene product to be retained within the host cell, this secretion signal sequence is omitted. In a similar manner, the expression vector can be constructed to include retention signal sequences for anchoring the prophylactic or therapeutic agent within the host cell plasma membrane. For example, membrane proteins have hydrophobic transmembrane regions that stop translocation of the protein in the membrane and do not allow the protein to be secreted. The construction of an expression vector including signal sequences for targeting a gene product to a particular location is deemed to be within the scope of one of skill in the art.

In an embodiment, vectors for mammalian host cell gene therapy are viruses, more preferably replication-deficient viruses (described in detail below). Exemplary viral vectors are derived from: Harvey Sarcoma Virus; Rous Sarcoma Virus, MPSV, Moloney murine leukemia virus and DNA viruses (e.g., adenovirus). See Temin, H., Retrovirus vectors for gene transfer, in GENE TRANSFER, Kucherlapati R, Ed., pp. 149-187 (Plenum 1986).

Replication-deficient retroviruses are capable of directing synthesis of virion proteins, but are incapable of making infectious particles. Accordingly, these genetically altered retroviral expression vectors have general utility for high-efficiency transduction of genes in cultured cells, and specific utility for use in the method of the present invention. Such retroviruses further have utility for the efficient transduction of genes into host cells in vivo. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in Kriegler, M. GENE TRANSFER AND EXPRESSION, A LABORATORY MANUAL, W. H. Freeman Co., NY (1990) and Murray, E. J., ed. METHODS IN MOLECULAR BIOLOGY, Vol. 7, Humana Press Inc., Clifton, N.J. (1991).

The major advantage of using retroviruses for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types (see e.g., Hilberg et al., Proc. Natl. Acad. Sci. USA 84: 5232-5236 (1987); Holland et al., Proc. Natl. Acad. Sci. USA 84: 8662-8666 (1987); Valerio et al., Gene 84: 419-427 (1989)). In vivo gene therapy using replication-deficient retroviral vectors to deliver a therapeutically effective amount of a therapeutic agent can be efficacious if the efficiency of transduction is high and/or the number of target cells available for transduction is high.

Yet another viral candidate useful as an expression vector for transformation of mammalian host cells is the adenovirus, a double-stranded DNA virus. The adenovirus is frequently responsible for respiratory tract infections in humans and thus appears to have an avidity for the epithelium of the respiratory tract (Straus, S., THE ADENOVIRUS, H. S. Ginsberg, Editor, Plenum Press, NY, p.451-496 (1984)). Moreover, the adenovirus is infective in a wide range of cell types, including, for example, muscle and epithelial cells (Larrick, J. W. and Burck, K. L., GENE THERAPY. APPLICATION OF MOLECULAR BIOLOGY, Elsevier Science Publishing Co., Inc., NY, p.71-104 (1991)). The adenovirus also has been used as an expression vector in muscle cells in vivo (Quantin, B., et al., Proc. Natl. Acad. Sci. USA 89: 2581-2584 (1992)).

Like the retrovirus, the adenovirus genome is adaptable for use as an expression vector for gene therapy, i.e., by removing the genetic information that controls production of the virus itself (Rosenfeld, M. A., et al., Science 252:431-434 (1991)). Because the adenovirus functions in an extrachromosomal fashion, the recombinant adenovirus does not have the theoretical problem of insertional mutagenesis.

Thus, as will be apparent to one skilled in the art, a variety of suitable viral expression vectors are available for transferring exogenous genetic material into mammalian host cells. The selection of an appropriate expression vector to express an agent for the identification, prevention or treatment of microbial infection amenable to gene replacement therapy and the optimization of the conditions for insertion of the selected expression vector into the cell are within the scope of one of skill in the art without the need for undue experimentation.

In an alternative embodiment, the expression vector is in the form of a plasmid, which is transferred into the target host cells by one of a variety of methods: physical (e.g., microinjection (Capecchi, M. R., Cell 22: 479-488 (1980)), electroporation (Andreason, G. L. and Evans, G. A. Biotechniques 6: 650-660 (1988)), scrape loading, microparticle bombardment (Johnston, S. A., Nature 346: 776-777 (1990)) or by cellular uptake as a chemical complex (e.g., calcium or strontium co-precipitation, complexation with lipid, complexation with ligand) (METHODS IN MOLECULAR BIOLOGY, Vol. 7, GENE TRANSFER AND EXPRESSION PROTOCOLS, Ed. E. J. Murray, Humana Press (1991)). Several commercial products are available for cationic liposome complexation including Lipofectin (Life Technologies, Inc., Gaithersburg, Md.) (Felgner, P. L., et al., Proc. Natl. Acad. Sci. 84: 7413-7417 (1987)) and Transfectam™ (ProMega, Madison, Wis.) (Behr, J. P., et al., Proc. Natl. Acad. Sci. USA 86: 6982-6986 (1989); Loeffler, J. P., et al., J. Neurochem. 54: 1812-1815 (1990)). However, the efficiency of transfection by these methods is highly dependent on the nature of the target cell and accordingly, the conditions for optimal transfection of nucleic acids into host cells using the above-mentioned procedures must be optimized. Such optimization is within the scope of one of skill in the art.

In an embodiment, the preparation of genetically modified host cells contains an amount of cells sufficient to deliver a prophylactically or therapeutically effective dose of a signal transduction mediator of the present invention to the recipient in situ. The determination of an effective dose of the prophylactic or therapeutic agent for a known microbial infection is within the scope of one of skill in the art. Thus, in determining the effective dose, the skilled artisan would consider the condition of the patient, the severity of the condition, as well as the results of clinical studies of the prophylactic or therapeutic agent being administered.

If the genetically modified host cells are not already present in a pharmaceutically acceptable carrier, they are placed in such a carrier prior to administration to the recipient. Such pharmaceutically acceptable carriers include, for example, isotonic saline and other buffers as appropriate to the patient and therapy. The genetically modified cells are administered by, for example, intraperitoneal injecting or implanting the cells or a graft or capsule containing the cells in a host cell-compatible site of the recipient. As used herein, host cell-compatible site refers to a structure, cavity or fluid of the recipient into which the genetically modified cell(s), host cell graft, or encapsulated host cell expression system can be implanted, without triggering adverse physiological consequences. Representative host cell-compatible sites include, for example, the peritoneal, pleural and pericardial cavities. Preferably, the host cell-compatible site communicates with the lymphatic system, thereby enabling delivery of the therapeutic agent to the vascular system.

In one embodiment, the host cell-compatible site may be denuded prior to implanting the cells. Exemplary denuding methods include but are not limited to: (1) injection of distilled water into the site (e.g., the peritoneal cavity) for 20 minutes, followed by scraping off a portion of the epithelial layer; (2) injection of 0.1% buffered trypsin for 20 minutes followed by scraping; (3) removal of epithelial cells by gentle scraping with a cell scraper and (4) touching a piece of Gelfilm (Upjohn, Kalamazoo, Mich.) to the endothelium.

The genetically modified host cells are implanted in a host cell-compatible site, alone or in combination with other genetically modified host cells. Thus, the instant invention embraces a method for modifying the epithelial system of a recipient by using a mixture of genetically modified host cells, such that a first modified cell expresses a first prophylactic or therapeutic agent of the present invention and a second modified cell expresses a second prophylactic or therapeutic agent. Other genetically modified cell types (e.g., hepatocytes, smooth muscle cells, fibroblasts, glial cells, mesothelial cells or keratinocytes) can be added, together with the genetically altered epithelial cells, to produce expression of a complex set of introduced genes. Moreover, more than one recombinant gene can be introduced into each genetically modified cell on the same or different vectors, thereby allowing the expression of multiple prophylactic or therapeutic agents of the present invention by a single cell.

The instant invention further embraces an epithelial cell graft. The graft comprises a plurality of the above-described genetically modified cells attached to a support that is suitable for implantation into a mammalian recipient, preferably into the oral cavity. The support can be formed of a natural or synthetic material. According to another aspect of the invention, an encapsulated host cell expression system is provided. The encapsulated system includes a capsule suitable for implantation into a mammalian recipient and a plurality of the above-described genetically modified host cells contained therein. The capsule can be formed of a synthetic or naturally-occurring material. The formulation of such capsules is known to one of ordinary skill in the art. In contrast to the host cells that are directly implanted into the mammalian recipient (i.e., implanted in a manner such that the genetically modified cells are in direct physical contact with the host cell-compatible site), the encapsulated cells remain isolated (i.e., not in direct physical contact with the site) following implantation. Thus, the encapsulated host cell system is not limited to a capsule including genetically-modified non-immortalized host cells, but may contain genetically modified immortalized host cells.

The invention has been disclosed broadly and illustrated in reference to representative embodiments described above. Those skilled in the art will recognize that various modifications can be made to the present invention without departing from the spirit and scope thereof.

We claim:

1. An isolated nucleic acid comprising a nucleotide sequence selected from the group consisting of SEQ ID NOs: 2, 3, and 5, wherein the nucleotide sequence encodes a polypeptide capable of catalyzing the transfer of a phosphate group from a donor molecule to an acceptor molecule.

2. A vector comprising a nucleotide sequence selected from the group consisting of SEQ ID NOs: 2, 3, and 5, wherein the nucleotide sequence encodes a polypeptide capable of catalyzing the transfer of a phosphate group from a donor molecule to an acceptor molecule.

3. A host cell transformed with the vector of claim 2.

4. The host cell of claim 3, wherein said host cell produces a polypeptide capable of catalyzing the transfer of a phosphate group from a donor molecule to an acceptor molecule.

5. An isolated nucleic acid capable of hybridizing under high stringency conditions to a nucleotide sequence selected from the group consisting of SEQ ID NOs: 2, 3, and 5.

6. The isolated nucleic acid of claim 5, wherein said isolated nucleic acid is capable of inhibiting the regulation of signal transduction.

7. The isolated nucleic acid of claim 5, wherein said nucleic acid is capable of inhibiting said transfer of said phosphate group from said donor molecule to said acceptor molecule.

8. A method of preventing or treating disease in a mammal comprising administering to said mammal syngeneic cells transformed with the vector of claim 2, wherein said transformed syngeneic cells produce a polypeptide capable of regulating signal transduction.

9. The method of claim 8, wherein said mammal is a human.

10. The method of claim 8, wherein said disease is cardiac disease.

11. The method of claim 8, wherein the syngeneic cells produces a polypeptide capable of catalyzing the transfer of a phosphate group from a donor molecule to an acceptor molecule.

12. A method of preventing or treating disease in a mammal comprising administering to said mammal syngeneic cells transformed with the vector of claim 2, wherein said transformed syngeneic cells produce a polypeptide capable of catalyzing the transfer of a phosphate group from a donor molecule to an acceptor molecule.

13. The method of claim 12, wherein said mammal is a human.

14. The method of claim 12, wherein said disease is cardiac disease.


SEQUENCE LISTING (9 sequences)

SEQ ID NO: 1 - protein, 1351 aa, Homo sapiens
GVQFSQYGSPEFVSPEIIQQNPVSEASDIWAMGVISYLSLTCSSPFAGESDRATLLNVLEGRVSWSSPMAAHLSEDAKDFIKATLQRAPQARPSAAQCLSHPWFLKSMPAEEAHFINTKQLKFLLARSRWQRSLMSYKSILVMRSIPELLRGPPDSPSLGVARHLCRDTGGSSSSSSSSDNELAPFARAKSLPPSPVTHSPLLHPRGFLRPSASLPEEAEASERSTEAPAPPASPEGAGPPAAQGCVPRHSVIRSLFYHQAGESPEHGALAPGSRRHPARRRHLLKGGYIAGALPGLREPLMEHRVLEEEAAREEQATLLAKAPSFETALRLPASGTHLAPGHSHSLEHDSPSTPRPSSEACGEAQRLPSAPSGGAPIRDMGHPQGSKQLPSTGGHPGTAQPERPSPDSPWGQPAPFCHPKQGSAPQEGCSPHPAVAPCPPGSFPPGSCKEAPLVPSSPFLGQPQAPPAPAKASPPLDSKMGPGDISLPGRPKPGPCSSPGSASQASSSQVSSLRVGSSQVGTEPGPSLDAEGWTQEAEDLSDSTPTLQRPQEQATMRKFSLGGRGGYAGVAGYGTFAFGGDAGGMLGQGPMWARIAWAVSQSEEEEQEEARAESQSEEQQEARAESPLPQVSARPVPEVGRAPTRSSPEPTPWEDIGQVSLVQIRDLSGDAEAADTISLDISEVDPAYLNLSDLYDIKYLPFEFMIFRKVPKSAQPEPPSPMAEEELAEFPEPTWPWPGELGPHAGLEITEESEDVDALLAEAAVGRKRKWSSPSRSLFHFPGRHLPLDEPAELGLRERVKASVEHISRILKGRPEGLEKEGPPRKKPGLASFRLSGLKSWDRAPTFLRELSDETVVLGQSVTLACQVSAQPAAQATWSKDGAPLESSSRVLISATLKNFQLLTILVVVAEDLGVYTCSVSNALGTVTTTGVLRKAERPSSSPCPDIGEVYADGVLLVWKPVESYGPVTYIVQCSLEGGSWTTLASDIFDCCYLTSKLSRGGTYTFRTACVSKAGMGPYSSPSEQVLLGGPSHLASEEESQGRSAQPLPSTKTFAFQTQIQRGRFSVVRQCWEKASGRALAAKIIPYHPKDKTAVLREYEALKGLRHPHLAQLHAAYLSPRHLVLILELCSGPELLPCLAERASYSESEVKDYLWQMLSATQYLHNQHILHLDLRSENMIITEYNLLKVVDLGNAQSLSQEKVLPSDKFKDYLETMAPELLEGQGAVPQTDIWAIGVTAFIMLSAEYPVSSEGARDLQRGLRKGLVRLSRCYAGLSGGAVAFLRSTLCAQPWGRPCASSCLQCPWLTEEGPACSRPAPVTFPTARLRVFVRNREKRRALLYKRHNLAQVR

SEQ ID NO: 2 - DNA, 4175 bp, Homo sapiens
  CDS (1)...(4053)
ggagtgcagttcagccagtacggctcccctgagttcgtctcccccgagatcatccagcagaaccctgtgagcgaagcctccgacatttgggccatgggtgtcatctcctacctcagcctgacctgctcatccccatttgccggcgagagtgaccgtgccaccctcctgaacgtcctggaggggcgcgtgtcatggagcagccccatggctgcccacctcagcgaagacgccaaagacttcatcaaggctacgctgcagagagcccctcaggcccggcctagtgcggcccagtgcctctcccacccctggttcctgaaatccatgcctgcggaggaggcccacttcatcaacaccaagcagctcaagttcctcctggcccgaagtcgctggcagcgttccctgatgagctacaagtccatcctggtgatgcgctccatccctgagctgctgcggggcccacccgacagcccctccctcggcgtagcccggcacctctgcagggacactggtggctcctccagttcctcctcctcctctgacaacgagctcgccccatttgcccgggctaagtcactgccaccctccccggtgacacactcaccactgctgcacccccggggcttcctgcggccctcggccagcctgcctgaggaagccgaggccagtgagcgctccaccgaggccccagctccgcctgcatctcccgagggtgccgggccaccggccgcccagggctgcgtgccccggcacagcgtcatccgcagcctgttctaccaccaggcgggtgagagccctgagcacggggccctggccccggggagcaggcggcacccggcccggcggcggcacctgctgaagggcgggtacattgcgggggcgctgccaggcctgcgcgagccactgatggagcaccgcgtgctggaggaggaggccgccagggaggagcaggccaccctcctggccaaagccccctcattcgagactgccctccggctgcctgcctctggcacccacttggcccctggccacagccactccctggaacatgactctccgagcaccccccgcccctcctcggaggcctgcggtgaggcacagcgactgccttcagccccctccgggggggcccctatcagggacatggggcaccctcagggctccaagcagcttccatccactggtggccacccaggcactgctcagccagagaggccatccccggacagcccttgggggcagccagcccctttctgccaccccaagcagggttctgccccccaggagggctgcagcccccacccagcagttgccccatgccctcctggctccttccctccaggatcttgcaaagaggcccccttagtaccctcaagccccttcttgggacagccccaggcaccccctgcccctgccaaagcaagccccccattggactctaagatggggcctggagacatctctcttcctgggaggccaaaacccggcccctgcagttccccagggtcagcctcccaggcgagctcttcccaagtgagctccctcagggtgggctcctcccaggtgggcacagagcctggcccctccctggatgcggagggctggacccaggaggctgaggatctgtccgactccacacccaccttgcagcggcctcaggaacaggcgaccatgcgcaagttctccctgggtggtcgcgggggctacgcaggcgtggctggctatggcacctttgcctttggtggagatgcagggggcatgctggggcaggggcccatgtgggccaggatagcctgggctgtgtcccagtcagaggaggaggagcaggaggaggccagggctgagtcccagtcggaggagcagcaggaggccagggctgagagcccactgccccaggtcagtgcaaggcctgtgcctgaggtcggcagggctcccaccaggagctctccagagcccaccccatgggaggacatcgggcaggtctccctggtgcagatccgggacctgtcaggtgatgcggaggcggccgacacaatatccctggacatttccgaggtggaccccgcctacctcaacctctcagacctgtacgatatcaagtacctcccattcgagtttatgatcttcaggaaagtccccaagtccgctcagccagagccgccctcccccatggctgaggaggagctggccgagttcccggagcccacgtggccctggccaggtgaactgggcccccacgcaggcctggagatcacagaggagtcagaggatgtggacgcgctgctggcagaggctgccgtgggcaggaagcgcaagtggtcctcgccgtcacgcagcctcttccacttccctgggaggcacctgccgctggatgagcctgcagagctggggctgcgtgagagagtgaaggcctccgtggagcacatctcccggatcctgaagggcaggccggaaggtctggagaaggaggggccccccaggaagaagccaggccttgcttccttccggctctcaggtctgaagagctgggaccgagcgccgacattcctaagggagctctcagatgagactgtggtcctgggccagtcagtgacactggcctgccaggtgtcagcccagccagctgcccaggccacctggagcaaagacggagcccccctggagagcagcagccgtgtcctcatctctgccaccctcaagaacttccagcttctgaccatcctggtggtggtggctgaggacctgggtgtgtacacctgcagcgtgagcaatgcgctggggacagtgaccaccacgggcgtcctccggaaggcagagcgcccctcatcttcgccatgcccggatatcggggaggtgtacgcggatggggtgctgctggtctggaagcccgtggaatcctacggccctgtgacctacattgtgcagtgcagcctagaaggcggcagctggaccacactggcctccgacatctttgactgctgctacctgaccagcaagctctcccggggtggcacctacaccttccgcacggcatgtgtcagcaaggcaggaatgggtccctacagcagcccctcggagcaagtcctcctgggagggcccagccacctggcctctgaggaggagagccaggggcggtcagcccaacccctgcccagcacaaagaccttcgcattccagacacagatccagaggggccgcttcagcgtggtgcggcaatgctgggagaaggccagcgggcgggcgctggccgccaagatcatcccctaccaccccaaggacaagacagcagtgctgcgcgaatacgaggccctcaagggcctgcgccacccgcacctggcccagctgcacgcagcctacctcagcccccggcacctggtgctcatcttggagctgtgctctgggcccgagctgctcccctgcctggccgagagggcctcctactcagaatccgaggtgaaggactacctgtggcagatgttgagtgccacccagtacctgcacaaccagcacatcctgcacctggacctgaggtccgagaacatgatcatcaccgaatacaacctgctcaaggtcgtggacctgggcaatgcacagagcctcagccaggagaaggtgctgccctcagacaagttcaaggactacctagagaccatggctccagagctcctggagggccagggggctgttccacagacagacatctgggccatcggtgtgacagccttcatcatgctgagcgccgagtacccggtgagcagcgagggtgcacgcgacctgcagagaggactgcgcaaggggctggtccggctgagccgctgctacgcggggctgtccgggggcgccgtggccttcctgcgcagcactctgtgcgcccagccctggggccggccctgcgcgtccagctgcctgcagtgcccgtggctaacagaggagggcccggcctgttcgcggcccgcgcccgtgaccttccctaccgcgcggctgcgcgtcttcgtgcgcaatcgcgagaagagacgcgcgctgctgtacaagaggcacaacctggcccaggtgcgctgagggtcgccccggccacacccttggtctccccgctgggggtcgctgcagacgcgccaataaaaacgcacagccgggcgagaaaaaaaaaaaaaaaaaaaaaaaaaaaaaaaaaaaaaaaa

SEQ ID NO: 3 - DNA, 5007 bp, Homo sapiens
  CDS (97)...(4926)
ctgctctgggacgtccacagccacgtggtcagagagaccacacagaggacctacacataccaggccatcgacacgcacaccgcacggcccccatccatgcaggtaaccatcgaggatgtgcaggcacagacaggcggaacggcccaattcgaggctatcattgagggcgacccacagccctcggtgacctggtacaaggacagcgtccagctggtggacagcacccggcttagccagcagcaagaaggcaccacatactccctggtgctgaggcatgtggcctcgaaggatgccggcgtttacacctgcctggcccaaaacactggtggccaggtgctctgcaaggcagagctgctggtgcttgggggggacaatgagccggactcagagaagcaaagccaccggaggaagctgcactccttctatgaggtcaaggaggagattggaaggggcgtgtttggcttcgtaaaaagagtgcagcacaaaggaaacaagatcttgtgcgctgccaagttcatccccctacggagcagaactcgggcccaggcatacagggagcgagacatcctggccgcgctgagccacccgctggtcacggggctgctggaccagtttgagacccgcaagaccctcatcctcatcctggagctgtgctcatccgaggagctgctggaccgcctgtacaggaagggcgtggtgacggaggccgaggtcaaggtctacatccagcagctggtggaggggctgcactacctgcacagccatggcgttctccacctggacataaagccctctaacatcctgatggtgcatcctgcccgggaagacattaaaatctgcgactttggctttgcccagaacatcaccccagcagagctgcagttcagccagtacggctcccctgagttcgtctcccccgagatcatccagcagaaccctgtgagcgaagcctccgacatttgggccatgggtgtcatctcctacctcagcctgacctgctcatccccatttgccggcgagagtgaccgtgccaccctcctgaacgtcctggaggggcgcgtgtcatggagcagccccatggctgcccacctcagcgaagacgccaaagacttcatcaaggctacgctgcagagagcccctcaggcccggcctagtgcggcccagtgcctctcccacccctggttcctgaaatccatgcctgcggaggaggcccacttcatcaacaccaagcagctcaagttcctcctggcccgaagtcgctggcagcgttccctgatgagctacaagtccatcctggtgatgcgctccatccctgagctgctgcggggcccacccgacagcccctccctcggcgtagcccggcacctctgcagggacactggtggctcctccagttcctcctcctcctctgacaacgagctcgccccatttgcccgggctaagtcactgccaccctccccggtgacacactcaccactgctgcacccccggggcttcctgcggccctcggccagcctgcctgaggaagccgaggccagtgagcgctccaccgaggccccagctccgcctgcatctcccgagggtgccgggccaccggccgcccagggctgcgtgccccggcacagcgtcatccgcagcctgttctaccaccaggcgggtgagagccctgagcacggggccctggccccggggagcaggcggcacccggcccggcggcggcacctgctgaagggcgggtacattgcgggggcgctgccaggcctgcgcgagccactgatggagcaccgcgtgctggaggaggaggccgccagggaggagcaggccaccctcctggccaaagccccctcattcgagactgccctccggctgcctgcctctggcacccacttggcccctggccacagccactccctggaacatgactctccgagcaccccccgcccctcctcggaggcctgcggtgaggcacagcgactgccttcagccccctccgggggggcccctatcagggacatggggcaccctcagggctccaagcagcttccatccactggtggccacccaggcactgctcagccagagaggccatccccggacagcccttgggggcagccagcccctttctgccaccccaagcagggttctgccccccaggagggctgcagcccccacccagcagttgccccatgccctcctggctccttccctccaggatcttgcaaagaggcccccttagtaccctcaagccccttcttgggacagccccaggcaccccctgcccctgccaaagcaagccccccattggactctaagatggggcctggagacatctctcttcctgggaggccaaaacccggcccctgcagttccccagggtcagcctcccaggcgagctcttcccaagtgagctccctcagggtgggctcctcccaggtgggcacagagcctggcccctccctggatgcggagggctggacccaggaggctgaggatctgtccgactccacacccaccttgcagcggcctcaggaacaggcgaccatgcgcaagttctccctgggtggtcgcgggggctacgcaggcgtggctggctatggcacctttgcctttggtggagatgcagggggcatgctggggcaggggcccatgtgggccaggatagcctgggctgtgtcccagtcagaggaggaggagcaggaggaggccagggctgagtcccagtcggaggagcagcaggaggccagggctgagagcccactgccccaggtcagtgcaaggcctgtgcctgaggtcggcagggctcccaccaggagctctccagagcccaccccatgggaggacatcgggcaggtctccctggtgcagatccgggacctgtcaggtgatgcggaggcggccgacacaatatccctggacatttccgaggtggaccccgcctacctcaacctctcagacctgtacgatatcaagtacctcccattcgagtttatgatcttcaggaaagtccccaagtccgctcagccagagccgccctcccccatggctgaggaggagctggccgagttcccggagcccacgtggccctggccaggtgaactgggcccccacgcaggcctggagatcacagaggagtcagaggatgtggacgcgctgctggcagaggctgccgtgggcaggaagcgcaagtggtcctcgccgtcacgcagcctcttccacttccctgggaggcacctgccgctggatgagcctgcagagctggggctgcgtgagagagtgaaggcctccgtggagcacatctcccggatcctgaagggcaggccggaaggtctggagaaggaggggccccccaggaagaagccaggccttgcttccttccggctctcaggtctgaagagctgggaccgagcgccgacattcctaagggagctctcagatgagactgtggtcctgggccagtcagtgacactggcctgccaggtgtcagcccagccagctgcccaggccacctggagcaaagacggagcccccctggagagcagcagccgtgtcctcatctctgccaccctcaagaacttccagcttctgaccatcctggtggtggtggctgaggacctgggtgtgtacacctgcagcgtgagcaatgcgctggggacagtgaccaccacgggcgtcctccggaaggcagagcgcccctcatcttcgccatgcccggatatcggggaggtgtacgcggatggggtgctgctggtctggaagcccgtggaatcctacggccctgtgacctacattgtgcagtgcagcctagaaggcggcagctggaccacactggcctccgacatctttgactgctgctacctgaccagcaagctctcccggggtggcacctacaccttccgcacggcatgtgtcagcaaggcaggaatgggtccctacagcagcccctcggagcaagtcctcctgggagcgcccagccacctggcctctgaggaggagagccaggggcggtcagcccaacccctgcccagcacaaagaccttcgcattccagacacagatccagaggggccgcttcagcgtggtgcggcaatgctgggagaaggccagcgggcgggcgctggccgccaagatcatcccctaccaccccaaggacaagacagcagtgctgcgcgaatacgaggccctcaagggcctgcgccacccgcacctggcccagctgcacgcagcctacctcagcccccggcacctggtgctcatcttggagctgtgctctgggcccgagctgctcccctgcctggccgagagggcctcctactcagaatccgaggtgaaggactacctgtggcagatgttgagtgccacccagtacctgcacaaccagcacatcctgcacctggacctgaggtccgagaacatgatcatcaccgaatacaacctgctcaaggtcgtggacctgggcaatgcacagagcctcagccaggagaaggtgctgccctcagacaagttcaaggactacctagagaccatggctccagagctcctggagggccagggggctgttccacagacagacatctgggccatcggtgtgacagccttcatcatgctgagcgccgagtacccggtgagcagcgagggtgcacgcgacctgcagagaggactgcgcaaggggctggtccggctgagccgctgctacgcggggctgtccgggggcgccgtggccttcctgcgcagcactctgtgcgcccagccctggggccggccctgcgcgtccagctgcctgcagtgcccgtggctaacagaggagggcccggcctgttcgcggcccgcgcccgtgaccttccctaccgcgcggctgcgcgtcttcgtgcgcaatcgcgagaagagacgcgcgctgctgtacaagaggcacaacctggcccaggtgcgctgagggtcgccccggccacacccttggtctccccgctgggggtcgctgcagacgcgccaataaaaacgcacagccgggcga

SEQ ID NO: 4 - protein, 1610 aa, Homo sapiens
MQVTIEDVQAQTGGTAQFEAIIEGDPQPSVTWYKDSVQLVDSTRLSQQQEGTTYSLVLRHVASKDAGVYTCLAQNTGGQVLCKAELLVLGGDNEPDSEKQSHRRKLHSFYEVKEEIGRGVFGFVKRVQHKGNKILCAAKFIPLRSRTRAQAYRERDILAALSHPLVTGLLDQFETRKTLILILELCSSEELLDRLYRKGVVTEAEVKVYIQQLVEGLHYLHSHGVLHLDIKPSNILMVHPAREDIKICDFGFAQNITPAELQFSQYGSPEFVSPEIIQQNPVSEASDIWAMGVISYLSLTCSSPFAGESDRATLLNVLEGRVSWSSPMAAHLSEDAKDFIKATLQRAPQARPSAAQCLSHPWFLKSMPAEEAHFINTKQLKFLLARSRWQRSLMSYKSILVMRSIPELLRGPPDSPSLGVARHLCRDTGGSSSSSSSSDNELAPFARAKSLPPSPVTHSPLLHPRGFLRPSASLPEEAEASERSTEAPAPPASPEGAGPPAAQGCVPRHSVIRSLFYHQAGESPEHGALAPGSRRHPARRRHLLKGGYIAGALPGLREPLMEHRVLEEEAAREEQATLLAKAPSFETALRLPASGTHLAPGHSHSLEHDSPSTPRPSSEACGEAQRLPSAPSGGAPIRDMGHPQGSKQLPSTGGHPGTAQPERPSPDSPWGQPAPFCHPKQGSAPQEGCSPHPAVAPCPPGSFPPGSCKEAPLVPSSPFLGQPQAPPAPAKASPPLDSKMGPGDISLPGRPKPGPCSSPGSASQASSSQVSSLRVGSSQVGTEPGPSLDAEGWTQEAEDLSDSTPTLQRPQEQATMRKFSLGGRGGYAGVAGYGTFAFGGDAGGMLGQGPMWARIAWAVSQSEEEEQEEARAESQSEEQQEARAESPLPQVSARPVPEVGRAPTRSSPEPTPWEDIGQVSLVQIRDLSGDAEAADTISLDISEVDPAYLNLSDLYDIKYLPFEFMIFRKVPKSAQPEPPSPMAEEELAEFPEPTWPWPGELGPHAGLEITEESEDVDALLAEAAVGRKRKWSSPSRSLFHFPGRHLPLDEPAELGLRERVKASVEHISRILKGRPEGLEKEGPPRKKPGLASFRLSGLKSWDRAPTFLRELSDETVVLGQSVTLACQVSAQPAAQATWSKDGAPLESSSRVLISATLKNFQLLTILVVVAEDLGVYTCSVSNALGTVTTTGVLRKAERPSSSPCPDIGEVYADGVLLVWKPVESYGPVTYIVQCSLEGGSWTTLASDIFDCCYLTSKLSRGGTYTFRTACVSKAGMGPYSSPSEQVLLGAPSHLASEEESQGRSAQPLPSTKTFAFQTQIQRGRFSVVRQCWEKASGRALAAKIIPYHPKDKTAVLREYEALKGLRHPHLAQLHAAYLSPRHLVLILELCSGPELLPCLAERASYSESEVKDYLWQMLSATQYLHNQHILHLDLRSENMIITEYNLLKVVDLGNAQSLSQEKVLPSDKFKDYLETMAPELLEGQGAVPQTDIWAIGVTAFIMLSAEYPVSSEGARDLQRGLRKGLVRLSRCYAGLSGGAVAFLRSTLCAQPWGRPCASSCLQCPWLTEEGPACSRPAPVTFPTARLRVFVRNREKRRALLYKRHNLAQVR

SEQ ID NO: 5 - DNA, 7928 bp, Homo sapiens
  CDS (60)...(7847)
gaattcggcgcgccagatatcacacgtgccaaggggctggctcactggtgccgccccgaatgctggagaggttcacccccaagaaagtgaagaaaggctccagcatcaccttctctgtgaaggtagaaggacgcccggtgcccaccgtgcactggctcagggaggaggctgagagaggcgtgctgtggattggccctgacacaccgggctacaccgtggccagctctgcgcagcagcacagcctggtcctgctggacgtgggccggcagcaccagggcacctacacatgcattgccagcaacgctgccggccaggccctctgctccgccagcctgcacgtctcgggcctgcctaaggtggaggagcaggagaaagtgaaggaagcgctgatttccactttcctgcaggggaccacacaagccatctcagcacaggggttggaaactgcgagttttgctgaccttggtgggcagaggaaagaagagcctctggctgccaaggaggccctcggccacctgtccctcgctgaggtgggcacagaggagttcctgcagaaactgacctcccagatcactgagatggtatcggccaagatcacgcaggccaagctgcaggtgccaggaggtgacagtgatgaggactccaagacaccatctgcatccccccgccatggccgatcacggccatcctccagcatccaggagtcttcctcagagtcagaggacggcgatgcccgaggcgagatctttgacatctacgtggtcaccgctgactacctgcccctaggggctgagcaggatgccatcacgctgcgggaaggccagtatgtggaggtcctggatgcagcccacccactgcgctggcttgtccgcaccaagcccaccaagtccagcccctcacggcagggctgggtgtcaccagcctacctggacaggaggctcaagctgtcacctgagtggggggccgctgaggcccctgagttccctggggaggctgtgtctgaagacgaatacaaggcaaggctgagctctgtgatccaggagctgctgagttctgagcaggccttcgtggaggagctgcagttcctgcagagccaccacctgcagcacctggagcgctgcccccacgtgcccatagccgtggccggccagaaggcagtcatcttccgcaatgtgcgggacatcggccgcttccacagcagcttcctgcaggagttgcagcagtgcgacacggacgacgacgtggccatgtgcttcatcaagaaccaggcggcctttgagcagtacctggagttcctggtggggcgtgtgcaggctgagtcggtggtcgtcagcacggccatccaggagttctacaagaaatacgcggaggaggccctgttggcaggggacccctctcagcccccgccaccacctctgcagcactacctggagcagccagtggagcgggtgcagcgctaccaggccttgctgaaggagttgatccgcaacaaggcgcggaacagacagaactgcgcgctgctggagcaggcctatgccgtggtgtctgccctgccacagcgcgctgagaacaagctgcacgtgtccctcatggagaactacccaggcaccctggaggccctgggcgagcccatccgccagggccacttcatcgtgtgggagggtgcaccgggggcccgcatgccctggaagggccacaaccgtcacgtgttcctcttccgcaaccacctggtaatctgcaagccccggcgagactcccgcaccgataccgtcagctacgtgttccggaacatgatgaagctgagcagcatcgacctgaacgaccaggtggagggggatgaccgcgccttcgaggtgtggcaggagcgggaggactcggtgcgcaagtacctgctgcaggcacggacagccattatcaagagctcgtgggtgaaggagatctgtggcatccagcagcgtctggccctgcctgtgtggcggcccccggactttgaagaggagctggccgactgcacagccgagctgggtgagacagtcaagctggcctgccgcgtgacgggcacacccaagcctgtcatcagctggtacaaagatgggaaagcagtgcaggtggacccccaccacatcctcattgaagaccctgatggctcgtgtgcactcatcctggacagcctgaccggtgtggactctggccagtacatgtgcttcgcggccagcgccgctggcaactgcagtaccctgggcaagatcctggtgcaagtcccaccacggttcgtgaacaaggtccgggcctcaccctttgtggagggagaggacgcccagttcacctgcaccatcgaaggcgccccgtacccgcagatcaggtggtacaaggacggggccctgctgaccactggcaacaagttccagacactgagtgagcctcgcagcggcctgctagtgctggtgatccgggcggccagcaaggaggacctggggctctacgagtgtgagctggtgaaccggctgggctccgcgcgggctagtgcggagctgcgcattcagagccccatgctgcaggcccaggagcagtgtcacagggagcagctcgtggctgcagtggaagacaccaccctggagcgagcggaccaggaggtcacatctgtcctgaagagactgctgggccccaaggcgccaggcccctccacaggggacctcactggccctggcccctgccccaggggggcacccgcactccaggaaaccggctcccagcccccagtcaccggaacttcggaggcacctgccgtgcccccgagggtgccacagcccctcctccacgaaggcccagagcaggagccggaggccattgccagagcccaggaatggactgtgcccattcggatggagggtgcagcctggcccggggcaggcacaggggagctgctctgggacgtccacagccacgtggtcagagagaccacacagaggacctacacataccaggccatcgacacgcacaccgcacggcccccatccatgcaggtaaccatcgaggatgtgcaggcacagacaggcggaacggcccaattcgaggctatcattgagggcgacccacagccctcggtgacctggtacaaggacagcgtccagctggtggacagcacccggcttagccagcagcaagaaggcaccacatactccctggtgctgaggcatgtggcctcgaaggatgccggcgtttacacctgcctggcccaaaacactggtggccaggtgctctgcaaggcagagctgctggtgcttgggggggacaatgagccggactcagagaagcaaagccaccggaggaagctgcactccttctatgaggtcaaggaggagattggaaggggcgtgtttggcttcgtaaaaagagtgcagcacaaaggaaacaagatcttgtgcgctgccaagttcatccccctacggagcagaactcgggcccaggcatacagggagcgagacatcctggccgcgctgagccacccgctggtcacggggctgctggaccagtttgagacccgcaagaccctcatcctcatcctggagctgtgctcatccgaggagctgctggaccgcctgtacaggaagggcgtggtgacggaggccgaggtcaaggtctacatccagcagctggtggaggggctgcactacctgcacagccatggcgttctccacctggacataaagccctctaacatcctgatggtgcatcctgcccgggaagacattaaaatctgcgactttggctttgcccagaacatcaccccagcagagctgcagttcagccagtacggctcccctgagttcgtctcccccgagatcatccagcagaaccctgtgagcgaagcctccgacatttgggccatgggtgtcatctcctacctcagcctgacctgctcatccccatttgccggcgagagtgaccgtgccaccctcctgaacgtcctggaggggcgcgtgtcatggagcagccccatggctgcccacctcagcgaagacgccaaagacttcatcaaggctacgctgcagagagcccctcaggcccggcctagtgcggcccagtgcctctcccacccctggttcctgaaatccatgcctgcggaggaggcccacttcatcaacaccaagcagctcaagttcctcctggcccgaagtcgctggcagcgttccctgatgagctacaagtccatcctggtgatgcgctccatccctgagctgctgcggggcccacccgacagcccctccctcggcgtagcccggcacctctgcagggacactggtggctcctccagttcctcctcctcctctgacaacgagctcgccccatttgcccgggctaagtcactgccaccctccccggtgacacactcaccactgctgcacccccggggcttcctgcggccctcggccagcctgcctgaggaagccgaggccagtgagcgctccaccgaggccccagctccgcctgcatctcccgagggtgccgggccaccggccgcccagggctgcgtgccccggcacagcgtcatccgcagcctgttctaccaccaggcgggtgagagccctgagcacggggccctggccccggggagcaggcggcacccggcccggcggcggcacctgctgaagggcgggtacattgcgggggcgctgccaggcctgcgcgagccactgatggagcaccgcgtgctggaggaggaggccgccagggaggagcaggccaccctcctggccaaagccccctcattcgagactgccctccggctgcctgcctctggcacccacttggcccctggccacagccactccctggaacatgactctccgagcaccccccgcccctcctcggaggcctgcggtgaggcacagcgactgccttcagccccctccgggggggcccctatcagggacatggggcaccctcagggctccaagcagcttccatccactggtggccacccaggcactgctcagccagagaggccatccccggacagcccttgggggcagccagcccctttctgccaccccaagcagggttctgccccccaggagggctgcagcccccacccagcagttgccccatgccctcctggctccttccctccaggatcttgcaaagaggcccccttagtaccctcaagccccttcttgggacagccccaggcaccccctgcccctgccaaagcaagccccccattggactctaagatggggcctggagacatctctcttcctgggaggccaaaacccggcccctgcagttccccagggtcagcctcccaggcgagctcttcccaagtgagctccctcagggtgggctcctcccaggtgggcacagagcctggcccctccctggatgcggagggctggacccaggaggctgaggatctgtccgactccacacccaccttgcagcggcctcaggaacaggcgaccatgcgcaagttctccctgggtggtcgcgggggctacgcaggcgtggctggctatggcacctttgcctttggtggagatgcagggggcatgctggggcaggggcccatgtgggccaggatagcctgggctgtgtcccagtcagaggaggaggagcaggaggaggccagggctgagtcccagtcggaggagcagcaggaggccagggctgagagcccactgccccaggtcagtgcaaggcctgtgcctgaggtcggcagggctcccaccaggagctctccagagcccaccccatgggaggacatcgggcaggtctccctggtgcagatccgggacctgtcaggtgatgcggaggcggccgacacaatatccctggacatttccgaggtggaccccgcctacctcaacctctcagacctgtacgatatcaagtacctcccattcgagtttatgatcttcaggaaagtccccaagtccgctcagccagagccgccctcccccatggctgaggaggagctggccgagttcccggagcccacgtggccctggccaggtgaactgggcccccacgcaggcctggagatcacagaggagtcagaggatgtggacgcgctgctggcagaggctgccgtgggcaggaagcgcaagtggtcctcgccgtcacgcagcctcttccacttccctgggaggcacctgccgctggatgagcctgcagagctggggctgcgtgagagagtgaaggcctccgtggagcacatctcccggatcctgaagggcaggccggaaggtctggagaaggaggggccccccaggaagaagccaggccttgcttccttccggctctcaggtctgaagagctgggaccgagcgccgacattcctaagggagctctcagatgagactgtggtcctgggccagtcagtgacactggcctgccaggtgtcagcccagccagctgcccaggccacctggagcaaagacggagcccccctggagagcagcagccgtgtcctcatctctgccaccctcaagaacttccagcttctgaccatcctggtggtggtggctgaggacctgggtgtgtacacctgcagcgtgagcaatgcgctggggacagtgaccaccacgggcgtcctccggaaggcagagcgcccctcatcttcgccatgcccggatatcggggaggtgtacgcggatggggtgctgctggtctggaagcccgtggaatcctacggccctgtgacctacattgtgcagtgcagcctagaaggcggcagctggaccacactggcctccgacatctttgactgctgctacctgaccagcaagctctcccggggtggcacctacaccttccgcacggcatgtgtcagcaaggcaggaatgggtccctacagcagcccctcggagcaagtcctcctgggagcgcccagccacctggcctctgaggaggagagccaggggcggtcagcccaacccctgcccagcacaaagaccttcgcattccagacacagatccagaggggccgcttcagcgtggtgcggcaatgctgggagaaggccagcgggcgggcgctggccgccaagatcatcccctaccaccccaaggacaagacagcagtgctgcgcgaatacgaggccctcaagggcctgcgccacccgcacctggcccagctgcacgcagcctacctcagcccccggcacctggtgctcatcttggagctgtgctctgggcccgagctgctcccctgcctggccgagagggcctcctactcagaatccgaggtgaaggactacctgtggcagatgttgagtgccacccagtacctgcacaaccagcacatcctgcacctggacctgaggtccgagaacatgatcatcaccgaatacaacctgctcaaggtcgtggacctgggcaatgcacagagcctcagccaggagaaggtgctgccctcagacaagttcaaggactacctagagaccatggctccagagctcctggagggccagggggctgttccacagacagacatctgggccatcggtgtgacagccttcatcatgctgagcgccgagtacccggtgagcagcgagggtgcacgcgacctgcagagaggactgcgcaaggggctggtccggctgagccgctgctacgcggggctgtccgggggcgccgtggccttcctgcgcagcactctgtgcgcccagccctggggccggccctgcgcgtccagctgcctgcagtgcccgtggctaacagaggagggcccggcctgttcgcggcccgcgcccgtgaccttccctaccgcgcggctgcgcgtcttcgtgcgcaatcgcgagaagagacgcgcgctgctgtacaagaggcacaacctggcccaggtgcgctgagggtcgccccggccacacccttggtctccccgctgggggtcgctgcagacgcgccaataaaaacgcacagccgggcga

SEQ ID NO: 6 - protein, 2596 aa, Homo sapiens
MLERFTPKKVKKGSSITFSVKVEGRPVPTVHWLREEAERGVLWIGPDTPGYTVASSAQQHSLVLLDVGRQHQGTYTCIASNAAGQALCSASLHVSGLPKVEEQEKVKEALISTFLQGTTQAISAQGLETASFADLGGQRKEEPLAAKEALGHLSLAEVGTEEFLQKLTSQITEMVSAKITQAKLQVPGGDSDEDSKTPSASPRHGRSRPSSSIQESSSESEDGDARGEIFDIYVVTADYLPLGAEQDAITLREGQYVEVLDAAHPLRWLVRTKPTKSSPSRQGWVSPAYLDRRLKLSPEWGAAEAPEFPGEAVSEDEYKARLSSVIQELLSSEQAFVEELQFLQSHHLQHLERCPHVPIAVAGQKAVIFRNVRDIGRFHSSFLQELQQCDTDDDVAMCFIKNQAAFEQYLEFLVGRVQAESVVVSTAIQEFYKKYAEEALLAGDPSQPPPPPLQHYLEQPVERVQRYQALLKELIRNKARNRQNCALLEQAYAVVSALPQRAENKLHVSLMENYPGTLEALGEPIRQGHFIVWEGAPGARMPWKGHNRHVFLFRNHLVICKPRRDSRTDTVSYVFRNMMKLSSIDLNDQVEGDDRAFEVWQEREDSVRKYLLQARTAIIKSSWVKEICGIQQRLALPVWRPPDFEEELADCTAELGETVKLACRVTGTPKPVISWYKDGKAVQVDPHHILIEDPDGSCALILDSLTGVDSGQYMCFAASAAGNCSTLGKILVQVPPRFVNKVRASPFVEGEDAQFTCTIEGAPYPQIRWYKDGALLTTGNKFQTLSEPRSGLLVLVIRAASKEDLGLYECELVNRLGSARASAELRIQSPMLQAQEQCHREQLVAAVEDTTLERADQEVTSVLKRLLGPKAPGPSTGDLTGPGPCPRGAPALQETGSQPPVTGTSEAPAVPPRVPQPLLHEGPEQEPEAIARAQEWTVPIRMEGAAWPGAGTGELLWDVHSHVVRETTQRTYTYQAIDTHTARPPSMQVTIEDVQAQTGGTAQFEAIIEGDPQPSVTWYKDSVQLVDSTRLSQQQEGTTYSLVLRHVASKDAGVYTCLAQNTGGQVLCKAELLVLGGDNEPDSEKQSHRRKLHSFYEVKEEIGRGVFGFVKRVQHKGNKILCAAKFIPLRSRTRAQAYRERDILAALSHPLVTGLLDQFETRKTLILILELCSSEELLDRLYRKGVVTEAEVKVYIQQLVEGLHYLHSHGVLHLDIKPSNILMVHPAREDIKICDFGFAQNITPAELQFSQYGSPEFVSPEIIQQNPVSEASDIWAMGVISYLSLTCSSPFAGESDRATLLNVLEGRVSWSSPMAAHLSEDAKDFIKATLQRAPQARPSAAQCLSHPWFLKSMPAEEAHFINTKQLKFLLARSRWQRSLMSYKSILVMRSIPELLRGPPDSPSLGVARHLCRDTGGSSSSSSSSDNELAPFARAKSLPPSPVTHSPLLHPRGFLRPSASLPEEAEASERSTEAPAPPASPEGAGPPAAQGCVPRHSVIRSLFYHQAGESPEHGALAPGSRRHPARRRHLLKGGYIAGALPGLREPLMEHRVLEEEAAREEQATLLAKAPSFETALRLPASGTHLAPGHSHSLEHDSPSTPRPSSEACGEAQRLPSAPSGGAPIRDMGHPQGSKQLPSTGGHPGTAQPERPSPDSPWGQPAPFCHPKQGSAPQEGCSPHPAVAPCPPGSFPPGSCKEAPLVPSSPFLGQPQAPPAPAKASPPLDSKMGPGDISLPGRPKPGPCSSPGSASQASSSQVSSLRVGSSQVGTEPGPSLDAEGWTQEAEDLSDSTPTLQRPQEQATMRKFSLGGRGGYAGVAGYGTFAFGGDAGGMLGQGPMWARIAWAVSQSEEEEQEEARAESQSEEQQEARAESPLPQVSARPVPEVGRAPTRSSPEPTPWEDIGQVSLVQIRDLSGDAEAADTISLDISEVDPAYLNLSDLYDIKYLPFEFMIFRKVPKSAQPEPPSPMAEEELAEFPEPTWPWPGELGPHAGLEITEESEDVDALLAEAAVGRKRKWSSPSRSLFHFPGRHLPLDEPAELGLRERVKASVEHISRILKGRPEGLEKEGPPRKKPGLASFRLSGLKSWDRAPTFLRELSDETVVLGQSVTLACQVSAQPAAQATWSKDGAPLESSSRVLISATLKNFQLLTILVVVAEDLGVYTCSVSNALGTVTTTGVLRKAERPSSSPCPDIGEVYADGVLLVWKPVESYGPVTYIVQCSLEGGSWTTLASDIFDCCYLTSKLSRGGTYTFRTACVSKAGMGPYSSPSEQVLLGAPSHLASEEESQGRSAQPLPSTKTFAFQTQIQRGRFSVVRQCWEKASGRALAAKIIPYHPKDKTAVLREYEALKGLRHPHLAQLHAAYLSPRHLVLILELCSGPELLPCLAERASYSESEVKDYLWQMLSATQYLHNQHILHLDLRSENMIITEYNLLKVVDLGNAQSLSQEKVLPSDKFKDYLETMAPELLEGQGAVPQTDIWAIGVTAFIMLSAEYPVSSEGARDLQRGLRKGLVRLSRCYAGLSGGAVAFLRSTLCAQPWGRPCASSCLQCPWLTEEGPACSRPAPVTFPTARLRVFVRNREKRRALLYKRHNLAQVR

SEQ ID NO: 7 - protein, 871 aa, Homo sapiens
MGPGDISLPGRPKPGPCSSPGSASQASSSQVSSLRVGSSQVGTEPGPSLDAEGWTQEAEDLSDSTPTLQRPQEQATMRKFSLGGRGGYAGVAGYGTFAFGGDAGGMLGQGPMWARIAWAVSQSEEEEQEEARAESQSEEQQEARAESPLPQVSARPVPEVGRAPTRSSPEPTPWEDIGQVSLVQIRDLSGDAEAADTISLDISEVDPAYLNLSDLYDIKYLPFEFMIFRKVPKSAQPEPPSPMAEEELAEFPEPTWPWPGELGPHAGLEITEESEDVDALLAEAAVGRKRKWSSPSRSLFHFPGRHLPLDEPAELGLRERVKASVEHISRILKGRPEGLEKEGPPRKKPGLASFRLSGLKSWDRAPTFLRELSDETVVLGQSVTLACQVSAQPAAQATWSKDGAPLESSSRVLISATLKNFQLLTILVVVAEDLGVYTCSVSNALGTVTTTGVLRKAERPSSSPCPDIGEVYADGVLLVWKPVESYGPVTYIVQCSLEGGSWTTLASDIFDCCYLTSKLSRGGTYTFRTACVSKAGMGPYSSPSEQVLLGGPSHLASEEESQGRSAQPLPSTKTFAFQTQIQRGRFSVVRQCWEKASGRALAAKIIPYHPKDKTAVLREYEALKGLRHPHLAQLHAAYLSPRHLVLILELCSGPELLPCLAERASYSESEVKDYLWQMLSATQYLHNQHILHLDLRSENMIITEYNLLKVVDLGNAQSLSQEKVLPSDKFKDYLETMAPELLEGQGAVPQTDIWAIGVTAFIMLSAEYPVSSEGARDLQRGLRKGLVRLSRCYAGLSGGAVAFLRSTLCAQPWGRPCASSCLQCPWLTEEGPACSRPAPVTFPTARLRVFVRNREKRRALLYKRHNLAQVR

SEQ ID NO: 8 - protein, 548 aa, Rattus norvegicus
MAHISRILKGKPEGPEKEGPPRKKAGLASFRLSGLKGRDQAPSFLRELSDEAVVLGQSVTLACQVLAQPTAQATWSKDGALLESSGHLLISSTLKNFQLLTILVVTEEDLGTYTCCVSNPLGTAVTTGVLRKAERPSSSPRPEVGELYTDAVLLVWKPVESYGPVTYIVQCCIEGGSWTTLASDISDCCYLTGKLPRGGMYTFRTACVSKAGMGPYSSPSEQVLLGGPNHLASEEESSRGRPAQLLPSTKTFAFQTQIRRGRFSVVRQCREKASGRALAAKIVPYQPEDKTTVLREYEALKRLHHPHLAQLHAAYLSPRHLVLILELCSGPELLPSLAERDSYSESDVKDYLWQMLSATQYLHAQHILHLDLRSENMMVTEYNLLKVIDLGNAQSLSQEKVPPPENFKDYLETMAPELLEGQGAVPQTDIWAIGVTAFIMLSGEYPVSSEGTRDLQKGLRKGLIQLSRCYAGLSGGAVAFLQSSLCARPWGRPCASTCLQCGWLTEEGPTGSRPTPVTFPTARLRAFVREREKRRALLYKKHNLAQVR

SEQ ID NO: 9 - protein, 548 aa, Mus musculus
MAHISRILKGRPEGPEREGPPRKKAGLASFRLSGLKGRDQAPSFLRELSDEAVVLGQSVTLACQVLAQPTAQATWSKDGVLLESSGHLLISSTLKNFQLLTILVVKEEDLGTYTCCVSNPLGTAVTTGVLRKAERPSSSPRPEVGELYKDAVLLVWKPVESCGPVTYIVQCCIEGGSWTTLASDISDCCYLTGKLSRGGMYIFRTACVSKAGMGPYSSPSEQVLLGGPNHLASEEESSRGRPAQLLPSTKTFAFQMQIRRGRFSVVRQCREKASGRALAAKIVPYQPEDKTAVLREYEALKRLHHPHLAQLHAAYLSPRHLVLILELCSGPELLPSLAERESYSESDVKDYLWQMLSATQYLHAQHILHLDLRSENMMVTEYNLLKVIDLGNAQSLDQEKVPAPENFKDYLETMAPELLEGQGAVPQTDIWAIGVTAFIMLSGEYPESSEGTRDLQKGLRKGLIRLSRCYAGLSGGAVAFLQSSLCAQPWGRPCASTCLQCGWLTEEGPTGSRPTPVTFPTVRLRAFVREREKRRALLYKKHNLAQVR